(19) United States
(12) Patent Application Publication
(10) Pub. No.: US 2017/0258405 A1
(43) Pub. Date: Sep. 14, 2017

(54) MEASUREMENT DEVICE AND MEASUREMENT METHOD

(71) Applicant: SONY CORPORATION, Tokyo (JP)

(72) Inventor: HIDEO SATO, TOKYO (JP)

(21) Appl. No.: 15/113,144

(22) PCT Filed: Nov. 19, 2015
(86) PCT No.: PCT/JP2015/082549

§ 371 Date: Jul. 21, 2016

(30) Foreign Application Priority Data
Dec. 1, 2014 (JP) 2014-243198

(51) Int. Cl. A61B 5/00; A61B 5/024

(52) U.S. Cl. CPC A61B 5/721; A61B 5/02416; A61B 5/7257; A61B 5/725; A61B 5/0022

(57) ABSTRACT

The present technology relates to a measurement device and a measurement method that can improve the accuracy of measurement of pulse waves and pulses.

A body motion signal extracting unit extracts a body motion signal containing a component generated by body motion from a first band signal containing the components in a first frequency band of a first measurement signal acquired by illuminating a portion having a pulse with light of a predetermined wavelength. An arithmetic unit generates a pulse wave signal that is the differential signal between a second band signal and the body motion signal, the second band signal containing the components in a second frequency band of the first measurement signal. The present technology can be applied to devices that measure pulse waves and pulses, for example.

### TECHNICAL FIELD

The present technology relates to a measurement device and a measurement method, and more particularly, relates to a measurement device and a measurement method that are suitable for measuring pulse waves and pulses.

### BACKGROUND ART

Optical measurement devices that measure pulse waves and pulses are widely used these days (see Patent Document 1, for example).

### CITATION LIST

### Patent Document

Patent Document 1: JP 2008-538186 W

### SUMMARY OF THE INVENTION

### Problems to be Solved by the Invention

With an optical measurement device, however, it is extremely difficult to remove, from a measurement signal, a body motion component generated by body motion of the subject to be measured. Particularly, in a case where measurement is carried out at a portion with large motion, such as the surface of an arm, the body motion component is often larger than the pulse wave component generated in the pulse wave. This makes removal of the body motion component more difficult. As a result, the accuracy of measurement of pulse waves and pulses becomes lower.

In view of the above, the present technology aims to improve the accuracy of measurement of pulse waves and pulses.

### Solutions to Problems

A measurement device according to one aspect of the present technology includes: a body motion signal extracting unit that extracts a body motion signal containing a component generated by body motion from a first band signal containing the components in a first frequency band of a first measurement signal acquired by illuminating a portion having a pulse with light of a first wavelength; and an arithmetic unit that generates a pulse wave signal that is a differential signal between a second band signal and the body motion signal, the second band signal containing the components in a second frequency band of the first measurement signal.

The body motion signal extracting unit may predict and extract the body motion signal, using an autoregressive model.

The body motion signal extracting unit may generate the autoregressive model within the range of the fifth through twelfth orders, using the Yule-Walker's method.

The body motion signal extracting unit may set the order of the autoregressive model in accordance with the level of the first band signal.

The measurement device may further include: a body motion detecting unit that detects the body motion; and a measuring unit that measures a pulse wave frequency in accordance with the pulse wave signal or the second band signal, whichever is selected in accordance with a result of the detection of the body motion.

The body motion signal extracting unit may extract the body motion signal from a signal having attenuated frequency components in a band containing the measured value of the previous pulse wave frequency, the attenuated frequency components being of the frequency components of the first band signal.

The measuring unit may include: a frequency detecting unit that detects a first peak frequency that is the peak frequency of the pulse wave signal, and a second peak frequency that is the peak frequency of the second band signal; and a selecting unit that selects the pulse wave frequency in accordance with at least one of the result of the detection of the body motion and the measured value of the previous pulse wave frequency, the pulse wave frequency being the first peak frequency or the second peak frequency.

The frequency detecting unit may limit the frequency band in which the first peak frequency and the second peak frequency are to be detected, in accordance with the measured value of the previous pulse wave frequency.

The frequency detecting unit may detect the first peak frequency in accordance with a result of Fourier transform of the pulse wave signal subjected to padding with a sample of the value “0”, and detect the second peak frequency in accordance with a result of Fourier transform of the second band signal subjected to padding with a sample of the value “0”.

The measuring unit may include: a selecting unit that selects the pulse wave signal or the second band signal in accordance with the result of the detection of the body motion; and a frequency detecting unit that detects the pulse wave frequency that is the peak frequency of the signal selected by the selecting unit.

The frequency detecting unit may limit the frequency band in which the peak frequency is to be detected, in accordance with the measured value of the previous pulse wave frequency.

The frequency detecting unit may detect the peak frequency in accordance with a result of Fourier transform of a signal obtained by performing padding on the signal selected by the selecting unit with a sample of the value “0”.

The body motion detecting unit may detect the body motion in accordance with the frequency distribution of the body motion signal.

The body motion detecting unit may detect the body motion in accordance with the distribution of a combined vector of a third band signal and the first band signal, the third band signal containing the components in the first frequency band of a second measurement signal acquired by illuminating the portion having the pulse with light of a second wavelength.

The body motion detecting unit may detect the body motion in accordance with fluctuation of the first measurement signal and fluctuation of a second measurement signal acquired by illuminating the portion having the pulse with light of a second wavelength.

The measuring unit may calculate a pulse rate in accordance with the pulse wave frequency.

The measurement device may further include: a first filter that extracts the first band signal from the first measurement signal; and a second filter that extracts the second band signal from the first measurement signal. In this measurement device, the second frequency band may include the range of pulse wave frequencies to be measured, and the largest value in the second frequency band may be larger than the largest value in the first frequency band.

The measurement device may further include a filter that extracts the first band signal from the first measurement signal. In this measurement device, the first frequency band may be the same as the second frequency band and include the range of pulse wave frequencies to be measured, and the first band signal may be the same as the second band signal.

A measurement method according to the one aspect of the present technology includes: a body motion signal extraction step of extracting a body motion signal containing a component generated by body motion from a first band signal containing the components in a first frequency band of a first measurement signal acquired by illuminating a portion having a pulse with light of a predetermined wavelength; and an arithmetic step of generating a pulse wave signal that is a differential signal between a second band signal and the body motion signal, the second band signal containing the components in a second frequency band of the first measurement signal.

In the one aspect of the present technology, a body motion signal containing a component generated by body motion is extracted from a first band signal containing the components in a first frequency band of a first measurement signal acquired by illuminating a portion having a pulse with light of a predetermined wavelength, and a pulse wave signal that is a differential signal between a second band signal and the body motion signal is generated, the second band signal containing the components in a second frequency band of the first measurement signal.

### Effects of the Invention

According to one aspect of the present technology, pulse wave components can be extracted from measurement signals with high accuracy. According to the one aspect of the present technology, the accuracy of measurement of pulse waves and pulses can be increased.

### BRIEF DESCRIPTION OF DRAWINGS

FIG. 1 is graphs for explaining the relationship between the pulse wave and body motion.

FIG. 2 is an external view of an embodiment of a measurement device to which the present technology is applied.

FIG. 3 is a block diagram showing an example structure of the main unit of the measurement device.

FIG. 4 is a block diagram showing an example structure of a light receiving IC.

FIG. 5 is a timing chart for explaining an example operation of the light receiving IC.

FIG. 6 is a block diagram showing a first embodiment of the arithmetic processing unit of a measurement device.

FIG. 7 is a graph for explaining wavelengths of measurement light.

FIG. 8 is a flowchart for explaining a first embodiment of a pulse measurement process.

FIG. 9 is a graph for explaining examples of peak frequency detection ranges.

FIG. 10 is graphs showing examples of measured waveforms.

FIG. 11 is a graph for explaining a modification of a downsampling rate.

FIG. 12 is a block diagram showing a second embodiment of the arithmetic processing unit of a measurement device.

FIG. 13 is a flowchart for explaining a second embodiment of a pulse measurement process.

FIG. 14 is graphs showing a first example of measurement signals with respect to measurement light of respective wavelengths.

FIG. 15 is graphs showing a second example of measurement signals with respect to measurement light of respective wavelengths.

FIG. 16 is graphs showing example distributions of a combined vector.

FIG. 17 is a block diagram showing a third embodiment of the arithmetic processing unit of a measurement device.

FIG. 18 is a flowchart for explaining a third embodiment of a pulse measurement process.

FIG. 19 is graphs showing examples of measured waveforms.

FIG. 20 is graphs showing examples of envelopes of a measurement signal before and after band limitation.

FIG. 21 is graphs for explaining a modification of the peak frequency detection range.

FIG. 22 is a block diagram showing an example structure of a computer.

### MODES FOR CARRYING OUT THE INVENTION

The following is a description of modes (hereinafter referred to as embodiments) for carrying out the present technology. Explanation will be made in the following order.

### 1. Relationship Between Pulse Wave and Body Motion

### 2. First Embodiment

### 3. Second Embodiment

### 4. Third Embodiment

### 5. Modifications

### 1. Relationship Between Pulse Wave and Body Motion

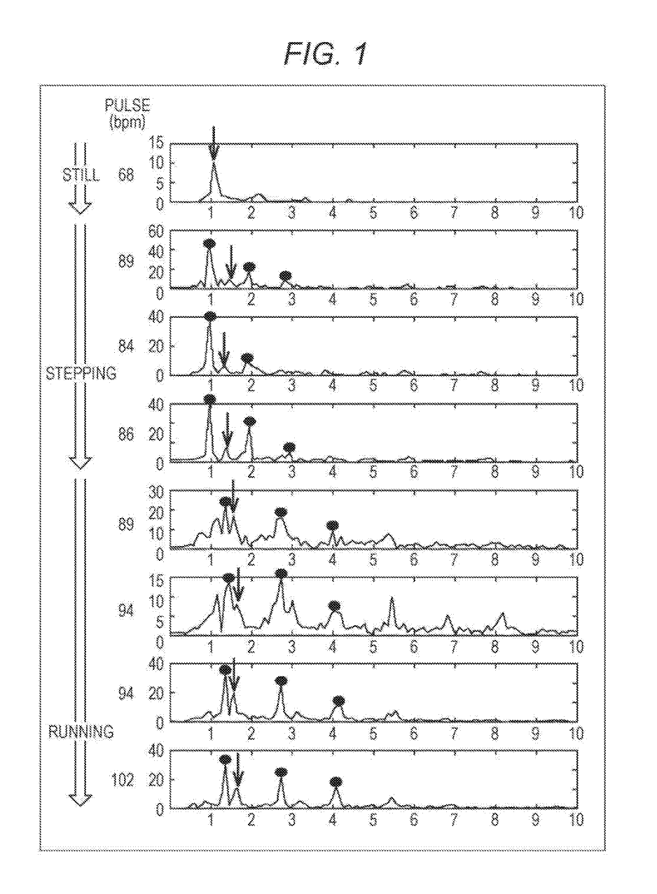

Each graph in FIG. 1 shows an example of a result of FFT (Fast Fourier Transform) frequency analysis of a signal obtained by measuring the pulse wave of an arm of a subject with an optical measurement device. In each graph, the abscissa axis indicates frequency, and the ordinate axis indicates spectrum intensity. The variations of the frequency distributions of measurement signals in a case where the subject first stands still, then starts stepping, and then starts running are sequentially shown, starting from the uppermost graph.

The uppermost graph in FIG. 1 shows the frequency distribution of a measurement signal at the time when the subject stands still. The second through fourth graphs from the top show the frequency distributions of measurement signals at the times when the subject is stepping. The fifth through eighth graphs from the top show the analysis results at the times when the subject is running. The pulses of the subject at the times of measurement of the measurement signals in the respective graphs from the top are 68 bpm (beats per minute), 89 bpm, 84 bpm, 86 bpm, 89 bpm, 94 bpm, 94 bpm, and 102 bpm.

The peak frequency indicated by an arrow in each graph is the frequency of the pulse wave of the subject (hereinafter referred to as the pulse wave frequency), and the frequency peaks indicated by black dots are the frequencies of body motion of the subject (hereinafter referred to as the body motion frequencies).

Normally, the fundamental frequency of a body motion component is slightly lower than the pulse wave frequency, as shown in this example. A body motion component contains a cyclic high overtone component. Furthermore, the energy of a body motion component is much greater than the energy of a pulse wave component. The frequency spectrum of a pulse wave component and the frequency spectrum of a body motion component become closer to each other, as the momentum becomes greater, and the pulse becomes faster.

In view of the above, it is extremely difficult for a comb-like filter or a bandpass filter (BPF) to separate pulse wave components from body motion components with high accuracy. Since the energy of a body motion component is much greater than the energy of a pulse wave component, it is very difficult to learn the pulse wave during rest, and extract the pulse wave from a measurement signal at a time of body motion. Also, an enormous amount of calculation is required to extract pulse wave components from measurement signals with higher accuracy. As a result, the resources for the CPU (Central Processing Unit), the memory, and the like, and the power consumption might increase. Also, the processing time might become longer, hindering real-time processing.

In the respective embodiments of the present technology described below, the influence of body motion components with such characteristics can be removed, and pulse waves and pulses can be measured with high accuracy.

### 2. First Embodiment

### [Example Structure of a Measurement Device 1]

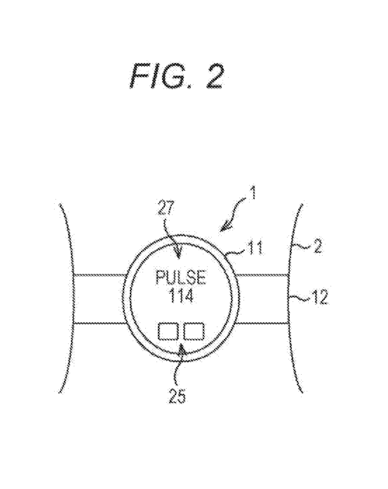

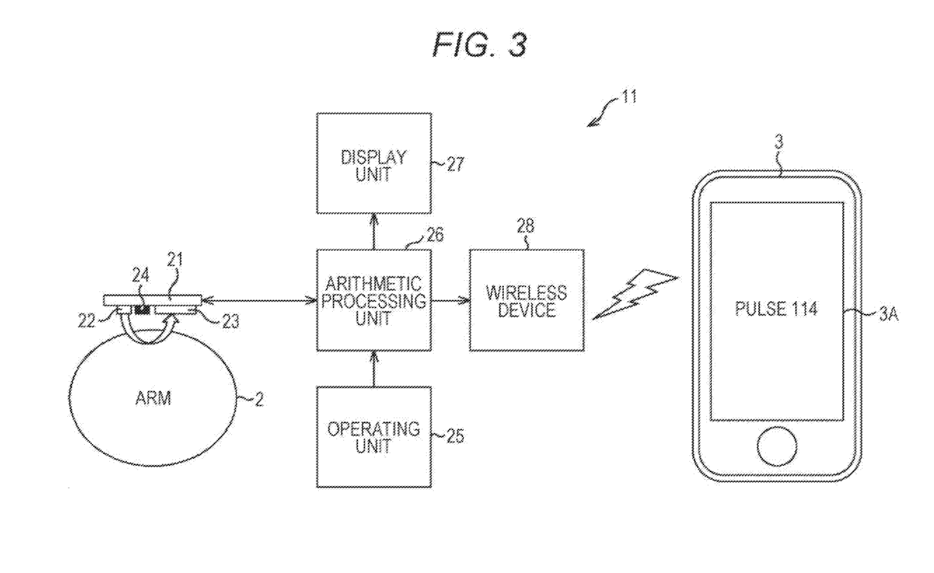

FIGS. 2 and 3 show an example structure of a measurement device 1 that is a first embodiment of a measurement device to which the present technology is applied. FIG. 2 shows an example structure of the exterior of the measurement device 1. FIG. 3 shows an example structure of a main unit 11 of the measurement device 1.

The measurement device 1 is a wristband-type measurement device that measures the pulse of a subject by an optical method. As shown in FIG. 2, the measurement device 1 includes a main unit 11 and a band 12, and the band 12 is attached to an arm (a wrist) 2 of the subject like a wristwatch. The main unit 11 illuminates the portion including the pulse of the arm 2 of the subject with measurement light of a predetermined wavelength, and measures the pulse of the subject in accordance with the intensity of returned light.

The main unit 11 is designed to include a substrate 21, an LED 22, a light receiving IC 23, a light shield 24, an operating unit 25, an arithmetic processing unit 26, a display unit 27, and a wireless device 28. The LED 22, the light receiving IC 23, and the light shield 24 are provided on the substrate 21.

Under the control of the light receiving IC 23, the LED 22 illuminates the portion including the pulse of the arm 2 of the subject with the measurement light of the predetermined wavelength.

The light receiving IC 23 receives light returned after the arm 2 is illuminated with the measurement light. The light receiving IC 23 generates a digital measurement signal indicating the intensity of the returned light, and supplies the generated measurement signal to the arithmetic processing unit 26.

The light shield 24 is provided between the LED 22 and the light receiving IC 23 on the substrate 21. The light shield 24 prevents direct entrance of the measurement light from the LED 22 into the light receiving IC 23.

The operating unit 25 is formed with various operating members such as buttons and switches, and is provided on a surface or the like of the main unit 11. The operating unit 25 is used in operating the measurement device 1, and supplies a signal indicating the contents of the operation to the arithmetic processing unit 26.

In accordance with the measurement signal supplied from the light receiving IC 23, the arithmetic processing unit 26 performs arithmetic processing to measure the pulse of the subject. The arithmetic processing unit 26 supplies a result of the pulse measurement to the display unit 27 and the wireless device 28.

The display unit 27 is formed with s display device such as an LCD (Liquid Crystal Display), and is provided on a surface of the main unit 11. The display unit 27 displays the result of the measurement of the subject's pulse and the like.

The wireless device 28 transmits the result of the measurement of the subject's pulse to an external device through wireless communication by a predetermined method. For example, as shown in FIG. 3, the wireless device 28 transmits the result of the measurement of the subject's pulse to a smartphone 3, and causes the screen 3A of the smartphone 3 to display the measurement result. The wireless device 28 can use any appropriate communication method.

### [Example Structure of the Light Receiving IC 23]

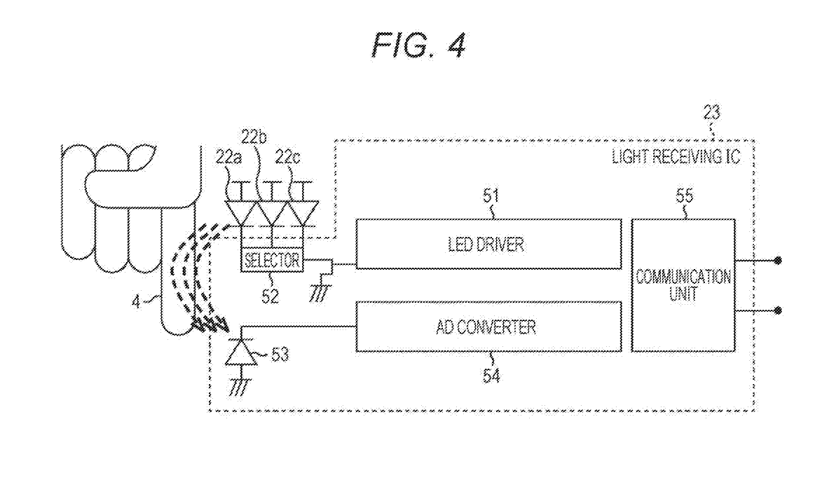

FIG. 4 shows an example structure of the light receiving IC 23. In this example case, the LED 22 of the measurement device 1 is formed with the three LEDs: LEDs 22a through 22c. The LEDs 22a through 22c emit measurement light of different wavelengths from one another.

The light receiving IC 23 is designed to include an LED driver 51, a selector 52, a light receiving element 53, an AD converter 54, and a communication unit 55.

The LED driver 51 supplies a drive signal to the LEDs 22a through 22c via the selector 52, and controls switching on and off of the LEDs 22a through 22c and the amounts of light to be emitted from the LEDs 22a through 22c.

The selector 52 selects, from among the LEDs 22a through 22c, the LED to which the drive signal is to be supplied from the LED driver 51, and supplies the drive signal to the selected LED.

The light receiving element 53 receives the light returned after the arm 2 is illuminated with the measurement light from the LEDs 22a through 22c. The light receiving element 53 supplies the measurement signal that is an analog electrical signal indicating the intensity of the received light, to the AD converter 54.

The AD converter 54 performs measurement signal sampling at a predetermined sampling frequency, and converts the analog measurement signal into a digital measurement signal. The AD converter 54 supplies the digital measurement signal to the communication unit 55. The sampling frequency of the AD converter 54 may be 200 Hz to 220 Hz, for example. In the example case described below, the sampling frequency of the AD converter 54 is 200 Hz.

The communication unit 55 communicates with the arithmetic processing unit 26 by a predetermined cable communication method, and transmits/receives the measurement signal, various control signals, and the like. The communication unit 55 can use any appropriate communication method, such as I2C.

As shown in FIG. 4, the light receiving IC 23 can be used to measure the pulse at a site (a finger 4, an earlobe (not shown), or the like) other than the arm 2 of the subject.

### [Example Operation of the Light Receiving IC 23]

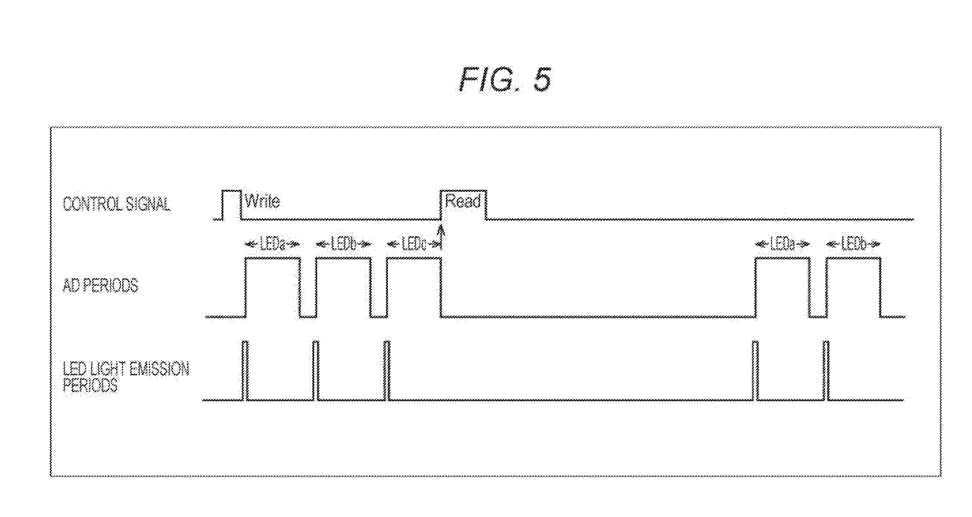

Referring now to the timing chart in FIG. 5, an example operation of the light receiving IC 23 is described. The first graph in FIG. 5 indicates the timings of the control signal to be supplied from the arithmetic processing unit 26 to the communication unit 55 of the light receiving IC 23. The second graph indicates the operation periods of the AD converter 54. The third graph indicates the light emission periods of the LEDs 22a through 22c. In these graphs, the LED 22a is shown as LEDa, the LED 22b is shown as LEDb, and the LED 22c is shown as LEDc.

The light receiving IC 23 starts operating when a Write signal is supplied from the arithmetic processing unit 26 to the communication unit 55.

Specifically, the LED driver 51 first supplies the drive signal to the LED 22a via the selector 52, and causes the LED 22a to emit pulse-like measurement light. The light receiving element 53 receives the light returned after the arm 2 is illuminated with the measurement light from the LED 22a. The light receiving element 53 supplies a measurement signal that is an analog electrical signal indicating the intensity of the received light (this measurement signal will be hereinafter referred to as the measurement signal a), to the AD converter 54. The AD converter 54 performs sampling on the analog measurement signal a, and performs AD conversion to convert the analog measurement signal a into a digital measurement signal a. The AD converter 54 supplies the digital measurement signal a to the communication unit 55.

After the AD conversion on the measurement signal a is completed, the LED driver 51 supplies the drive signal to the LED 22b via the selector 52, and causes the LED 22b to emit pulse-like measurement light. The light receiving element 53 receives the light returned after the arm 2 is illuminated with the measurement light from the LED 22b. The light receiving element 53 supplies a measurement signal that is an analog electrical signal indicating the intensity of the received light (this measurement signal will be hereinafter referred to as the measurement signal b), to the AD converter 54. The AD converter 54 performs sampling on the analog measurement signal b, and performs AD conversion to convert the analog measurement signal b into a digital measurement signal b. The AD converter 54 supplies the digital measurement signal b to the communication unit 55.

After the AD conversion on the measurement signal b is completed, the LED driver 51 supplies the drive signal to the LED 22c via the selector 52, and causes the LED 22c to emit pulse-like measurement light. The light receiving element 53 receives the light returned after the arm 2 is illuminated with the measurement light from the LED 22c. The light receiving element 53 supplies a measurement signal that is an analog electrical signal indicating the intensity of the received light (this measurement signal will be hereinafter referred to as the measurement signal c), to the AD converter 54. The AD converter 54 performs sampling on the analog measurement signal c, and performs AD conversion to convert the analog measurement signal c into a digital measurement signal c. The AD converter 54 supplies the digital measurement signal c to the communication unit 55.

When a Read signal is supplied from the arithmetic processing unit 26, the communication unit 55 supplies the measurement signals a through c to the arithmetic processing unit 26.

After a predetermined period of idle time has passed since the supply of the measurement signals a through c, the above described series of processes from the light emission from the LED 22a to the supply of the measurement signals a through c to the arithmetic processing unit 26 are performed.

The above series of processes are repeated for a predetermined period of time or a predetermined number of times, or are repeated until a stop command is input from the arithmetic processing unit 26, for example.

Although the measurement light has three kinds of wavelengths in FIGS. 4 and 5, one or two kinds of measurement light wavelengths may be set, or four or more kinds of measurement light wavelengths may be set. In the first embodiment, an example case where the measurement light has one kind of wavelength is described below.

[Example Structure of an Arithmetic Processing Unit 26a]

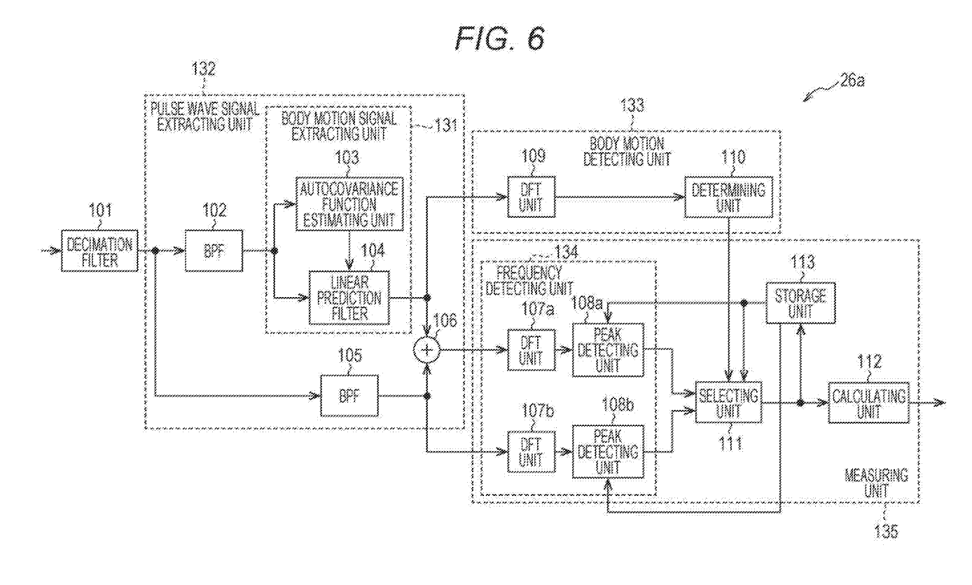

FIG. 6 shows an example structure of an arithmetic processing unit 26a that is the first embodiment of the arithmetic processing unit 26 of the measurement device 1. The arithmetic processing unit 26a is designed to include a decimation filter 101, a bandpass filter (BPF) 102, an autocovariance function estimating unit 103, a linear prediction filter 104, a bandpass filter (BPF) 105, an arithmetic unit 106, discrete Fourier transform (DFT) units 107a and 107b, peak detecting units 108a and 108b, a discrete Fourier transform (DFT) unit 109, a determining unit 110, a selecting unit 111, a calculating unit 112, and a storage unit 113.

The autocovariance function estimating unit 103 and the linear prediction filter 104 constitute a body motion signal extracting unit 131. The BPF 102, the BPF 105, the arithmetic unit 106, and the body motion signal extracting unit 331 constitute a pulse wave signal extracting unit 132. The DFT unit 109 and the determining unit 110 constitute a body motion detecting unit 133. The DFT units 107a and 107b, and the peak detecting units 108a and 108b constitute a frequency detecting unit 134. The selecting unit 111, the calculating unit 112, the storage unit 113, and the frequency detecting unit 134 constitute a measuring unit 135.

The decimation filter 101 performs downsampling on a measurement signal. The decimation filter 101 supplies the measurement signal after the downsampling, to the BPF 102 and the BPF 105.

The BPF 102 is formed with a zero-phase filter formed with two stages of third-order infinite impulse response (IIR) filters, for example. The BPF 102 passes the components in a predetermined frequency band (hereinafter referred to as the frequency band N) of the measurement signal, and blocks the components other than the components in the frequency band N. The BPF 102 also cancels out phase distortion by filtering the measurement signal with the third-order IIR filters of the first stage, and then filtering the measurement signal with the third-order IIR filters of the second stage in the reverse of the sampling order, for example. The third-order IIR filters of the second stage are the same as those of the first stage. The BPF 102 supplies a signal containing the extracted components in the frequency band N (this signal will be hereinafter referred to as the band signal N) to the autocovariance function estimating unit 103 and the linear prediction filter 104.

Since the BPF 102 is a zero-phase filter, temporal axis information such as body motion components is held in the extracted band signal N.

The autocovariance function estimating unit 103 estimates the autocovariance function of the body motion signal contained in the band signal N. The body motion signal is a signal containing the body motion components generated by body motion of the subject. The autocovariance function estimating unit 103 supplies a result of the estimation of the autocovariance function to the linear prediction filter 104.

The linear prediction filter 104 is formed with a linear prediction filter generated by a power spectrum density estimation algorithm according to the Yule-Walker's method, for example. For example, the linear prediction filter 104 determines parameters for the AR model (autocorrelation model) of a body motion signal according to the Yule-Walker's method, using the autocovariance function estimated by the autocovariance function estimating unit 103. As a result, the AR model of a body motion signal is generated. The linear prediction filter 104 predicts the body motion signal by using the generated AR model. If the body motion components are sufficiently greater than the pulse wave components, the linear prediction filter 104 extracts, from the band signal N, the body motion signal formed only with the body motion components not containing any low-level pulse wave components and noise components. The linear prediction filter 104 supplies the extracted body motion signal to the arithmetic unit 106 and the DFT unit 109.

Like the BPF 102, the BPF 105 is formed with a zero-phase filter formed with two stages of third-order infinite impulse response (IIR) filters, for example. The BPF 105 passes the components in a predetermined frequency band (hereinafter referred to as the frequency band W) of the measurement signal, and blocks the components other than the components in the frequency band W. The BPF 105 supplies a signal containing the extracted components in the frequency band W (this signal will be hereinafter referred to as the band signal W) to the arithmetic unit 106 and the DFT unit 107b.

Since the BPF 105 is a zero-phase filter, temporal axis information such as pulse wave components is held in the extracted band signal W.

The arithmetic unit 106 adds the inverted signal of the body motion signal to the band signal W, to calculate the difference between the band signal W and the body motion signal. The arithmetic unit 106 supplies the DFT unit 107a with a differential signal generated by calculating the difference between the band signal W and the body motion signal (this differential signal will be hereinafter referred to as the pulse wave signal).

The DFT unit 107a performs DFT on the pulse wave signal, and supplies a result of frequency analysis of the pulse wave signal to the peak detecting unit 108a.

In accordance with the result of the frequency analysis of the pulse wave signal, the peak detecting unit 108a detects the peak frequency of the pulse wave signal. In doing so, the peak detecting unit 108a limits the frequency band in which the peak frequency is to be detected, in accordance with the detected value of the previous pulse wave frequency stored in the storage unit 113. The peak detecting unit 108a supplies the detected value of the peak frequency of the pulse wave signal to the selecting unit 111.

The DFT unit 107b performs DFT on the band signal W, and supplies a result of frequency analysis of the band signal W to the peak detecting unit 108b.

In accordance with the result of the frequency analysis of the band signal W, the peak detecting unit 108b detects the peak frequency of the band signal W. In doing so, the peak detecting unit 108b limits the frequency band in which the peak frequency is to be detected, in accordance with the detected value of the previous pulse wave frequency stored in the storage unit 113. The peak detecting unit 108b supplies the detected value of the peak frequency of the band signal W to the selecting unit 111.

The DFT unit 109 performs DFT on the band signal N, and supplies a result of frequency analysis of the band signal N to the determining unit 110.

In accordance with the result of the frequency analysis of the band signal N, the determining unit 110 determines whether body motion hindering pulse measurement has been generated (this body motion will be hereinafter referred to as hindrance body motion). The determining unit 110 supplies the selecting unit 111 with a result of the determination as to generation of hindrance body motion.

In accordance with the result of the determination as to generation of hindrance body motion, and the measured value of the previous pulse wave frequency, the selecting unit 111 selects a pulse wave frequency that is the peak frequency of the pulse wave signal or the peak frequency of the band signal W. The selecting unit 111 supplies the calculating unit 112 with information indicating the selected pulse wave frequency, and stores the information into the storage unit 113.

In accordance with the pulse wave frequency, the calculating unit 112 calculates the pulse rate. The calculating unit 112 outputs the calculated pulse rate as a measurement result to the outside.

The storage unit 113 stores the measured values of the past pulse wave frequencies.

### [Wavelengths of Measurement Light]

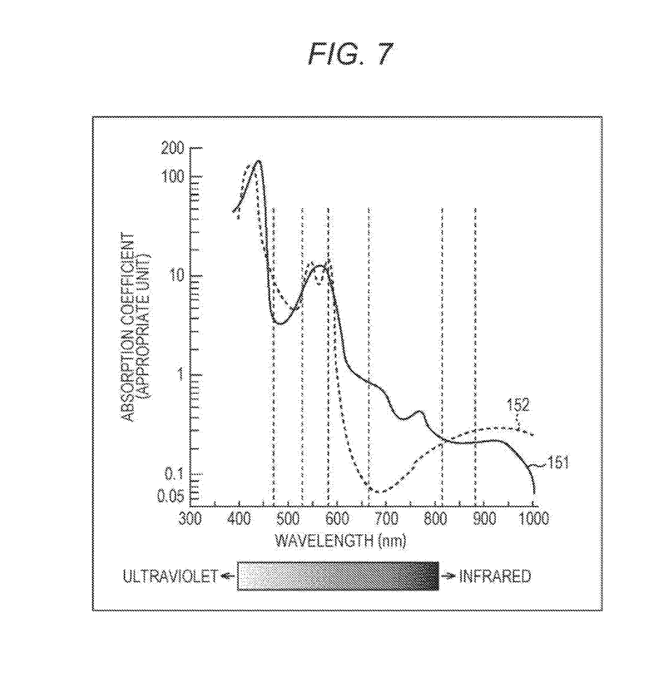

FIG. 7 shows the absorption characteristics relative to light in the respective wavebands of Hb (reduced hemoglobin) and HbO₂ (oxyhemoglobin) contained in blood. The abscissa axis indicates wavelength, and the ordinate axis indicates absorption coefficient. A curve 151 indicates the absorption characteristics of Hb, and a curve 152 indicates the absorption characteristics of HbO₂.

For example, 470-nm light (hereinafter referred to as the blue measurement light) or 660-nm light (hereinafter referred to as the red measurement light), which causes a large absorption coefficient difference between Hb and HbO₂ in the visible light region, is used as measurement light. Also, 530-nm light (hereinafter referred to as the green measurement light) or 585-nm light (hereinafter referred to as the yellow measurement light), with which the absorption coefficients of Hb and HbO₂ are substantially the same in the visible light region, is used as measurement light, for example. Also, 805-nm light with which the absorption coefficients of Hb and HbO₂ are substantially the same in the infrared region, or 880-nm light that causes a large absorption coefficient difference between Hb and HbO₂ in the infrared region is used as measurement light, for example.

### [First Embodiment of a Pulse Measurement Process]

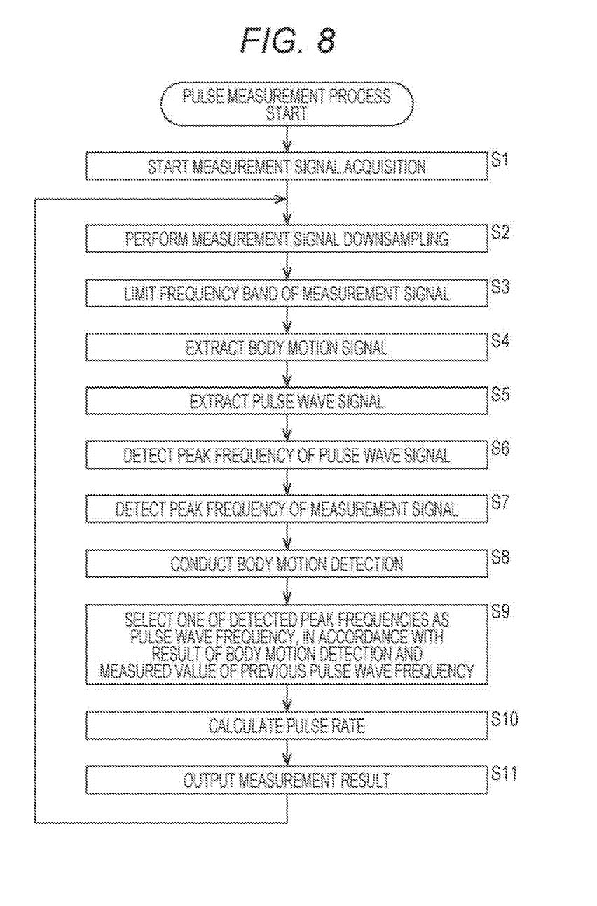

Referring now to the flowchart shown in FIG. 8, a first embodiment of a pulse measurement process to be performed by the measurement device 1 is described.

Pulse measurement is carried out at predetermined intervals (every 8 to 15 seconds, for example). In the example case described below, pulse measurement is carried out every eight seconds.

In step S1, the measurement device 1 starts measurement signal acquisition. Specifically, as described above with reference to FIG. 5, light emission from the LED 22 and light reception with the light receiving IC 23 are started. Also, the supply of a measurement signal indicating the intensity of received light from the light receiving IC 23 to the arithmetic processing unit 26 is started.

In step S2, the decimation filter 101 performs measurement signal downsampling in the frequency band in which pulse wave signal analysis is necessary. For example, the decimation filter 101 performs a decimation process on the measurement signal, and downsamples the number of samples of measurement signals at a predetermined rate. The decimation filter 101 supplies the measurement signal subjected to the downsampling, to the BPF 102 and the BPF 105.

In this example, the sampling frequency of measurement signals is 200 Hz, and one measurement period is eight seconds. Accordingly, the number of samples of measurement signals in one measurement period is 1600. In a case where the downsampling rate is 1/16, for example, the number of samples of measurement signals in one measurement period decreases from 1600 to 100.

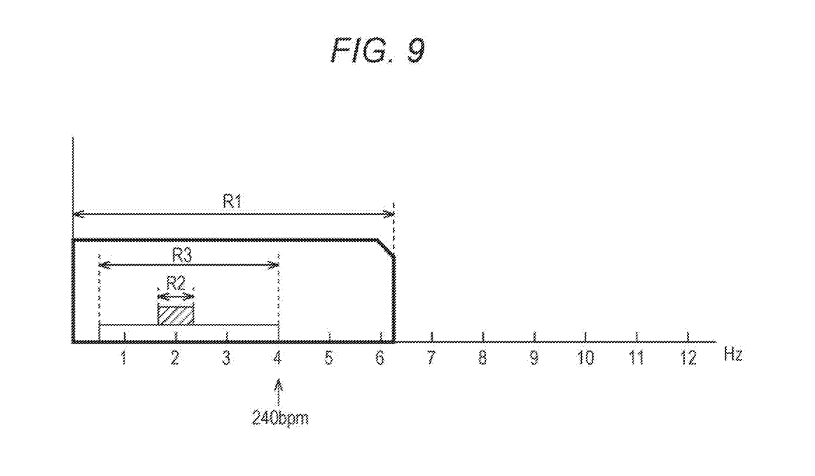

In this case, the sampling frequency of the measurement signal after the downsampling is 12.5 Hz (=200 Hz× 1/16). With the use of the measurement signal after the downsampling, frequency components up to 6.25 Hz can be detected, as indicated by a range R1 in FIG. 9.

Meanwhile, the highest pulse rate of a person is approximately 220 bpm, and therefore, 240 bpm should be sufficient as the highest pulse rate that can be measured with the measurement device 1. The pulse rate of 240 bpm is equivalent to 4.0 Hz in pulse wave frequency. In view of this, if 6.25-Hz frequency components can be detected with the measurement signal after the downsampling, the pulse wave frequency of a person can be measured with sufficient accuracy.

Also, as the downsampling is performed on the measurement signal, the amount of calculation to be performed thereafter can be reduced.

In the example case described below, the downsampling rate is set at 1/16.

In step S3, the pulse wave signal extracting unit 132 limits the frequency band of the measurement signal. Specifically, the BPF 102 extracts the components in the frequency band N from the measurement signal after the downsampling. The BPF 102 supplies the band signal N containing the extracted components in the frequency band N to the autocovariance function estimating unit 103 and the linear prediction filter 104.

The BPF 105 extracts the components in the frequency band W from the measurement signal after the downsampling. The BPF 105 supplies the band signal W containing the extracted components in the band signal W to the arithmetic unit 106 and the DFT unit 107b.

Here, the frequency band N and the frequency band W are set in accordance with the expected ranges of pulse wave frequencies and body motion frequencies. That is, the frequency band N and the frequency band W are set in accordance with the range of pulse wave frequencies to be measured, and the range of frequencies of body motion components to be extracted.

For example, the frequency band W is set so as to include at least the range of pulse wave frequencies to be measured. Meanwhile, the frequency band N is set so as to include at least the range of the frequencies of body motion components that are assumed to be in the frequency band W.

In a case where the range of pulse rates that can be measured with the measurement device 1 is 30 bpm to 240 bpm, for example, the equivalent range in pulse wave frequency is 0.5 Hz to 4.0 Hz. In this case, the minimum value of the frequency band W is set at 0.5 Hz or lower, and the maximum value is set at 4.0 Hz or higher.

Meanwhile, as described above with reference to FIG. 1, the fundamental frequency of body motion components is likely to be slightly lower than the pulse wave frequency. In this case, if the range of the fundamental frequencies of body motion components to be detected is 0.5 Hz to 2.5 Hz, the minimum value of the frequency band N is set at 0.5 Hz or lower, and the maximum value is set at 2.5 Hz or higher.

In the example case described below, the frequency band W is 0.5 Hz to 4.0 Hz, and the frequency band N is 0.5 Hz to 2.5 Hz. That is, the frequency band W in this case is a wider band than the frequency band N. More specifically, the maximum value of the frequency band W is set at a greater value than the maximum value of the frequency band N, and the minimum value of the frequency band W is set at the same value as the minimum value of the frequency band N.

In step S4, the body motion signal extracting unit 131 extracts the body motion signal. For example, the autocovariance function estimating unit 103 estimates the autocovariance function of the body motion signal included in the band signal N, in accordance with a predetermined number (eight, for example) of top samples among 100 samples in one measurement period of the band signal N. The autocovariance function estimating unit 103 supplies a result of the estimation of the autocovariance function to the linear prediction filter 104.

Using the estimated autocovariance function, the linear prediction filter 104 determines the parameters for the AR model of a body motion signal by the Yule-Walker's method, in accordance with the predetermined number (eight, for example) of top samples among the 100 samples in one measurement period of the band signal N. As a result, the AR model of a body motion signal is generated. Using the generated AR model, the linear prediction filter 104 predicts a body motion signal. In accordance with the result of the prediction of a body motion signal, the linear prediction filter 104 extracts the body motion signal by removing the pulse wave components and the noise components from the predetermined number (92, for example) of the remaining samples in one measurement period of the band signal N.

The order of the AR model of a body motion signal is now described. In a case where only the noise components are to be removed from the band signal N with high accuracy, the order of the AR model is preferably set at 20th to 30th order, for example. In such a case, however, not only the body motion components but also the pulse wave components are extracted from the band signal N.

If the order of the AR model is lowered, on the other hand, the accuracy of the noise component removal to be conducted by the linear prediction filter 104 becomes lower. If many body motion components are included in the band signal N, however, not only the noise components but also the pulse wave components much weaker than the body motion components can be removed. As a result, only the body motion components can be effectively extracted from the band signal N with a small amount of calculation.

In view of the above, the order of the AR model is set at the eighth, for example. Through an experiment, the eighth order was determined to be the order on which the body motion components can be extracted from the band signal N with high accuracy. However, the order of the AR model is not limited to the eighth. For example, in a case where the order of the AR model is set within the range of the fifth through twelfth orders, the body motion components can be extracted from the band signal N with high accuracy

The linear prediction filter 104 supplies the extracted body motion signal to the arithmetic unit 106 and the DFT unit 109.

If any body motion component is not included in the band signal N, or if the body motion components included in the band signal N are small, the pulse wave components are also extracted from the band signal N by the linear prediction filter 104, so that the pulse wave components are included in the body motion signal.

In step S5, the arithmetic unit 106 extracts the pulse wave signal. Specifically, the arithmetic unit 106 adds the inverted signal of the body motion signal to the band signal W, to calculate the difference between the band signal W and the body motion signal, and generate the pulse wave signal that is a differential signal. The arithmetic unit 106 supplies the pulse wave signal to the DFT unit 107a.

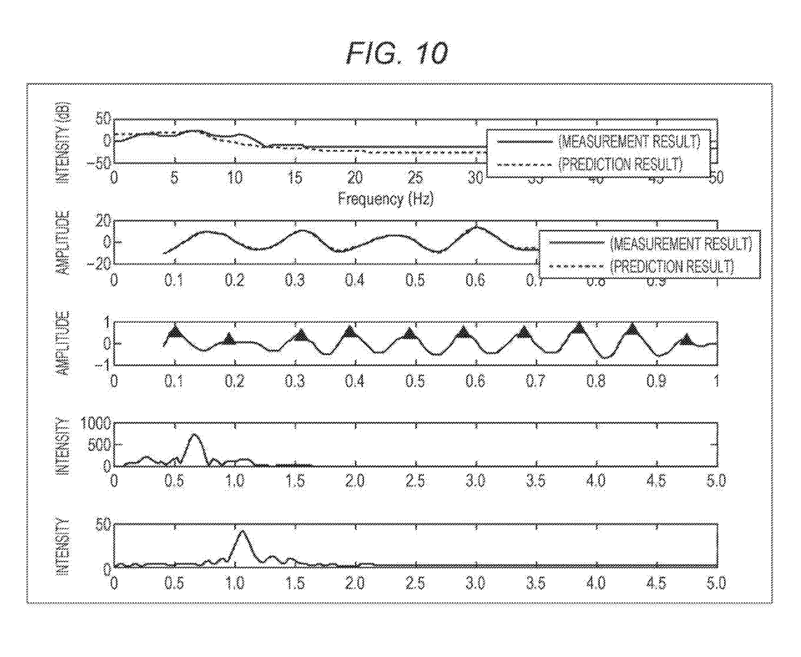

The uppermost graph in FIG. 10 shows an example of a result of measurement of the frequency distribution of the band signal W (the solid line), and an example of a result of prediction of the frequency distribution of the body motion signal using the AR model (the dashed line). In this graph, the abscissa axis indicates frequency, and the ordinate axis indicates spectrum intensity.

The second graph from the top in FIG. 10 shows an example of a result of measurement of the waveform of the band signal W (the solid line), and an example of a result of prediction of the waveform of the body motion signal using the AR model (the dashed line). In this graph, the abscissa axis indicates time, and the ordinate axis indicates amplitude.

The third graph from the top in FIG. 10 shows an example of the pulse wave signal that is the differential signal between the band signal W (the solid line) and the body motion signal (the dashed line) in the second graph from the top. In this graph, the abscissa axis indicates time, and the ordinate axis indicates amplitude.

The fourth graph from the top in FIG. 10 shows an example of the frequency distribution of the band signal W (the solid line) in the second graph from the top. In this graph, the abscissa axis indicates frequency, and the ordinate axis indicates spectrum intensity.

The lowermost graph in FIG. 10 shows an example of the frequency distribution of the pulse wave signal in the third graph from the top. In this graph, the abscissa axis indicates frequency, and the ordinate axis indicates spectrum intensity.

As shown in this example, the difference between the band signal W and the body motion signal is calculated, so that the body motion components are removed from the band signal W, and the pulse wave signal containing the pulse wave components is extracted. That is, the body motion components can be efficiently canceled out.

If any body motion component is not included in the band signal N, or if the body motion components included in the band signal N are small, the pulse wave components are included in the body motion signal, as described above. As a result, few pulse wave components are included in the pulse wave signal that is the differential signal between the band signal W and the body motion signal.

In step S6, the frequency detecting unit 134 detects the peak frequency of the pulse wave signal. Specifically, the DFT unit 107a performs padding with a predetermined number of samples of the value “0” between the samples of the pulse wave signals of the 100 samples in one measurement period. By doing so, the DFT unit 107a upsamples the sample signals to 1024 sample signals. The DFT unit 107a then performs DFT on the pulse wave signal after the padding, and supplies a result of pulse wave signal frequency analysis to the peak detecting unit 108a.

As the padding with samples of the value “0” is performed, and the number of pulse wave signal samples is increased to 1024 or 2048, the sampling frequency is made higher, and accordingly, the resolution of the pulse wave signal frequency analysis can be increased. Also, as the padding with samples of the value “0” is performed, DFT can be conducted with a sparse matrix, and the arithmetic processing can be performed at a higher speed.

In accordance with the result of the frequency analysis of the pulse wave signal, the peak detecting unit 108a detects the peak frequency of the pulse wave signal. At this point, the peak detecting unit 108a reads the measured value of the previous pulse wave frequency from the storage unit 113. In accordance with the measured value of the previous pulse wave frequency, the peak detecting unit 108a limits the frequency band in which the peak frequency is to be detected.

In a case where the measured value of the previous pulse wave frequency is 2 Hz, for example, the peak detecting unit 108a limits the frequency band in which the peak frequency is to be detected, to a detection range R2 of a predetermined bandwidth having its center at 2 Hz, as shown in FIG. 9. The detection range R2 is set as a narrower range than the bandwidth R3 of the frequency band W.

The peak detecting unit 108a detects the peak frequency of the pulse wave signal within the set detection range. The peak detecting unit 108a supplies the detected value of the peak frequency to the selecting unit 111.

As the peak frequency is detected within the detection range that is limited in accordance with the measured value of the previous pulse wave frequency as described above, the amount of calculation is reduced, and the possibility that a different peak frequency from the pulse wave frequency is detected can be lowered.

In step S7, the frequency detecting unit 134 detects the peak frequency of the measurement signal. Specifically, the DFT unit 107b carries out the same procedure as that carried out by the DFT unit 107a in step S6, to perform padding on the band signal W with samples of the value “0”, and then conduct DFT on the band signal W. Also, the peak detecting unit 108b carries out the same procedure as that carried out by the peak detecting unit 108a in step S6, to limit the detection range in accordance with the measured value of the previous pulse wave frequency, and then detect the peak frequency of the band signal W. As a result, the peak frequency within the detection range of the measurement signal is detected. The peak detecting unit 108b supplies the detected value of the peak frequency to the selecting unit 111.

In step S8, the body motion detecting unit 133 conducts body motion detection. Specifically, the DFT unit 109 performs DFT on the body motion signal, and supplies a result of frequency analysis of the body motion signal to the determining unit 110.

In accordance with the frequency distribution of the body motion signal, the determining unit 110 determines whether hindrance body motion has been generated. In a case where the frequency range of the body motion signal is equal to or wider than a predetermined range, the determining unit 110 determines that hindrance body motion has been generated. In a case where the frequency range of the body motion signal is narrower than the predetermined range, the determining unit 110 determines that no hindrance body motion has been generated.

The frequency range of the body motion signal is determined from the difference between the lowest frequency and the highest frequency among the frequencies at which the spectrum intensity of the body motion signal is equal to or higher than a predetermined threshold value, for example.

Alternatively, the determining unit 110 determines whether hindrance body motion has been generated, in accordance with the waveform of the frequency distribution of the body motion signal, for example. For example, using a discriminator obtained through advance machine learning, the determining unit 110 determines whether the waveform of the frequency distribution of the body motion signal is a waveform including a hindrance body motion component (a body motion component hindering pulse measurement). If the determining unit 110 determines, from a result of the discrimination, that a hindrance body motion component is included in the body motion signal, the determining unit 110 determines that hindrance body motion has been generated. If the determining unit 110 determines, from a result of the discrimination, that any hindrance body motion component is not included in the body motion signal, the determining unit 110 determines that no hindrance body motion has been generated.

The determining unit 110 then supplies the selecting unit 111 with a result of the determination as to generation of hindrance body motion.

The criteria for determining whether body motion of the subject is hindrance body motion are set through advance learning, experiments, or the like. As the criteria are appropriately set, weak body motion that does not hinder pulse measurement can be ignored.

In step S9, the selecting unit 111 selects a pulse wave frequency from the detected peak frequency, in accordance with a result of the body motion detection and the measured value of the previous pulse wave frequency. For example, the selecting unit 111 reads the measured value of the previous pulse wave frequency from the storage unit 113. In accordance with the measured value of the previous pulse wave frequency, the selecting unit 111 sets a selection reference range that is the widest possible range over which the pulse wave frequency can vary during the period from the previous measurement time to the current measurement time.

In a case where only either the peak frequency of the pulse wave signal or the peak frequency of the band signal W is within the selection reference range, the selecting unit 111 selects the peak frequency in the selection reference range as the pulse wave frequency.

In a case where both of the peak frequencies are within the selection reference range, or where neither of the peak frequencies is within the selection reference range, the selecting unit 111 selects the pulse wave frequency in accordance with a result of the determination as to generation of hindrance body motion.

Specifically, in a case where hindrance body motion has been generated, and many body motion components are included in the measurement signal, few pulse wave components remain in the body motion signal, as described above. Consequently, the pulse wave signal that is the differential signal between the band signal W and the body motion signal includes the pulse wave components, but include few body motion components. As a result, the peak frequency of the pulse wave signal is expected to be substantially equal to the pulse wave frequency of the subject. In view of this, when it is determined that hindrance body motion has been generated, the selecting unit 111 selects the peak frequency of the pulse wave signal as the pulse wave frequency. That is, the peak frequency of the pulse wave signal serves as the measured value of the current pulse wave frequency.

In a case where no hindrance body motion has been generated, and few body motion components are included in the measurement signal, most pulse wave components are not removed but remain in the body motion signal, as described above. Consequently, few pulse wave components are included in the pulse wave signal that is the differential signal between the band signal W and the body motion signal. Meanwhile, few body motion components are included in the band signal W. As a result, the peak frequency of the band signal W is expected to be substantially equal to the pulse wave frequency of the subject. In view of this, when it is determined that no hindrance body motion has been generated, the selecting unit 111 selects the peak frequency of the band signal W as the pulse wave frequency. That is, the peak frequency of the band signal W serves as the measured value of the current pulse wave frequency.

The selecting unit 111 supplies the calculating unit 112 with the information indicating the selected pulse wave frequency. The selecting unit 111 also stores the information indicating the selected pulse wave frequency into the storage unit 113. With this, the measured value of the current pulse frequency is stored into the storage unit 113.

In step S10, the calculating unit 112 calculates the pulse rate. For example, the calculating unit 112 calculates the pulse rate by multiplying the pulse wave frequency selected by the selecting unit 111, by 60.

In step S11, the calculating unit 112 outputs a result of the measurement. That is, the calculating unit 112 outputs the pulse rate calculated through the procedure in step S10 as a measurement result to the outside.

After that, the process returns to step S2, and the procedure in step S2 and the procedures thereafter are repeated.

In the above manner, influence of body motion can be eliminated, and the pulse wave and the pulse of the subject can be accurately measured with a small amount of calculation. For example, even when the subject engages in vigorous exercise, such as running, the pulse wave and the pulse of the subject can be accurately measured. In a case where measurement is carried out with the measurement device 1 attached to the subject for a long period of time, for example, the influence of body motion of the subject can be eliminated, and accurate measurement of the pulse wave and the pulse of the subject can be continued.

Also, as the amount of calculation is reduced, the power consumption by the measurement device 1 can be lowered. As a result, it becomes possible to continue measurement with the measurement device 1 attached to the subject for a long period of time, without any battery charging and replacement.

In the case described above, the downsampling rate is set at 1/16. However, the pulse wave frequency relative to the pulse rate of 240 bpm can be measured when the sampling frequency of the measurement signal after the downsampling is 8.0 Hz or higher. In a case where the sampling frequency of the measurement signal is 200 Hz, the downsampling rate can be lowered to 1/25.

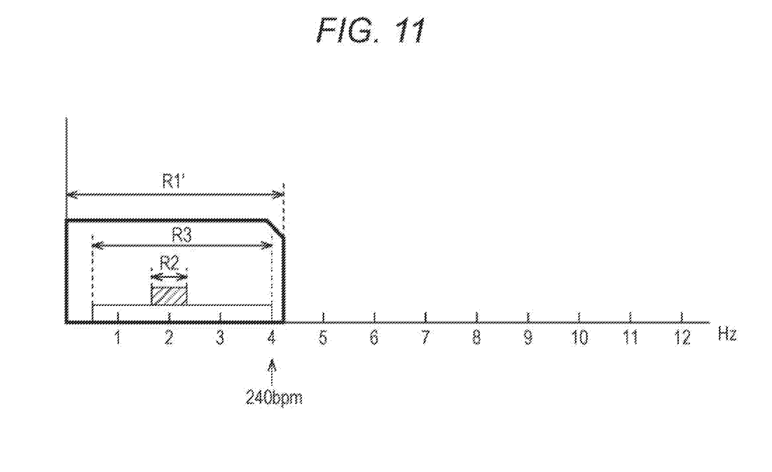

FIG. 11 shows an example case where the downsampling rate is set at 1/24. In this case, with the use of the measurement signal after the downsampling, frequency components up to 4.17 Hz (=200 Hz× 1/24÷2) can be detected, as indicated by a range R1′ in FIG. 11.

### 3. Second Embodiment

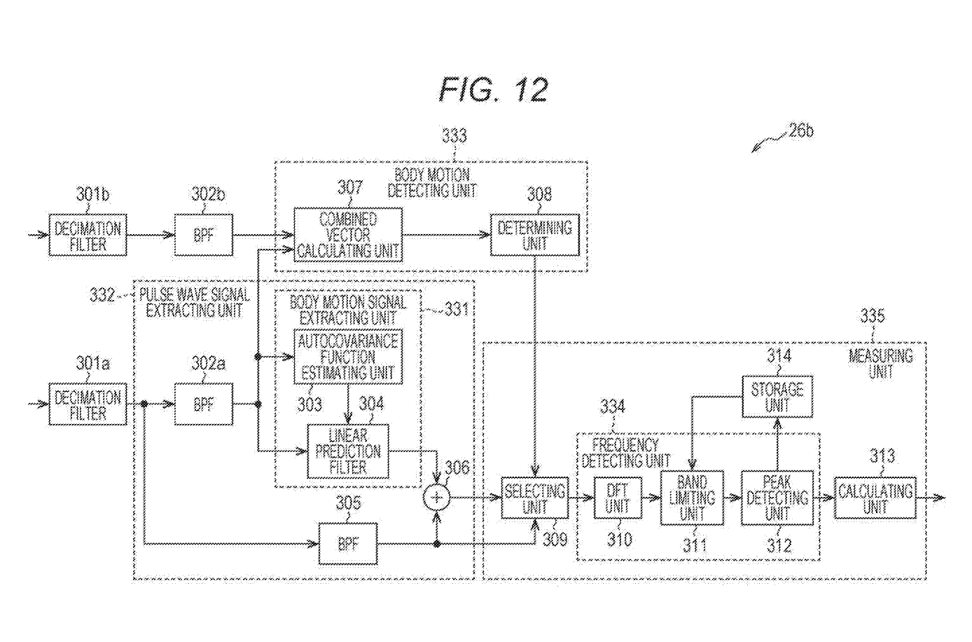

Referring now to FIGS. 12 through 16, a second embodiment of the present technology is described. The second embodiment differs from the first embodiment in the body motion detection method and the pulse wave frequency measurement method. In the second embodiment, two kinds of wavelength measurement light, measurement light 1 and measurement light 2, are used.

The measurement light 1 is blue measurement light of 470 nm in wavelength or green measurement light of 530 nm in wavelength, for example. In the example case described below, blue measurement light is used. A measurement signal 1 is measured with the measurement light 1.

The measurement light 2 is yellow measurement light of 585 nm in wavelength, for example. A measurement signal 2 is measured with the measurement light 2.

The sampling frequency of the measurement signal 1 and the measurement signal 2 is 200 Hz to 220 Hz, for example. In the example case described below, the sampling frequency of the measurement signal 1 and the measurement signal 2 is 200 Hz.

[Example Structure of an Arithmetic Processing Unit 26b]

In the second embodiment, an arithmetic processing unit 26b shown in FIG. 12, instead of the arithmetic processing unit 26a shown in FIG. 6, is used in a measurement device 1. The arithmetic processing unit 26b is designed to include decimation filters 301a and 301b, bandpass filters (BPFs) 302a and 302b, an autocovariance function estimating unit 303, a linear prediction filter 304, a bandpass filter (BPF) 305, an arithmetic unit 306, a combined vector calculating unit 307, a determining unit 308, a selecting unit 309, a discrete Fourier transform (DFT) unit 310, a band limiting unit 311, a peak detecting unit 312, a calculating unit 313, and a storage unit 314.

The autocovariance function estimating unit 303 and the linear prediction filter 304 constitute a body motion signal extracting unit 331. The BPF 302a, the BPF 305, the arithmetic unit 306, and the body motion signal extracting unit 331 constitute a pulse wave signal extracting unit 332. The combined vector calculating unit 307 and the determining unit 308 constitute a body motion detecting unit 333. The DFT unit 310, the band limiting unit 311, and the peak detecting unit 312 constitute a frequency detecting unit 334. The selecting unit 309, the calculating unit 313, the storage unit 314, and the frequency detecting unit 334 constitute a measuring unit 335.

The decimation filters 301a and 301b have the same functions as those of the decimation filter 101 shown in FIG. 6. The BPFs 302a and 302b have the same functions as those of the BPF 102 shown in FIG. 6. The autocovariance function estimating unit 303 has the same functions as those of the autocovariance function estimating unit 103 shown in FIG. 6. The linear prediction filter 304 has the same functions as those of the linear prediction filter 104 shown in FIG. 6. The BPF 305 has the same functions as those of the BPF 105 shown in FIG. 6. The arithmetic unit 306 has the same functions as those of the arithmetic unit 106 shown in FIG. 6. The DFT unit 310 has the same functions as those of the DFT units 107a and 107b shown in FIG. 6. The band limiting unit 311 and the peak detecting unit 312 achieve the same functions as those of the peak detecting units 108a and 108b shown in FIG. 6. The calculating unit 313 has the same functions as those of the calculating unit 112 shown in FIG. 6.

The decimation filter 301a performs downsampling on the measurement signal 1. The decimation filter 301a supplies the measurement signal 1 after the downsampling, to the BPF 302a and the BPF 305.

The decimation filter 301b performs downsampling on the measurement signal 2. The decimation filter 301b supplies the measurement signal 2 after the downsampling, to the BPF 302b.

The BPF 302a extracts the components in a frequency band N from the measurement signal 1, and supplies a signal containing the extracted components in the frequency band N (this signal will be hereinafter referred to as the band signal 1N) to the autocovariance function estimating unit 303, the linear prediction filter 304, and the combined vector calculating unit 307.

The BPF 302b extracts the components in the predetermined frequency band N from the measurement signal 2, and supplies a measurement signal containing the extracted components in the frequency band N (this measurement signal will be hereinafter referred to as the band signal 2N) to the combined vector calculating unit 307.

Like the autocovariance function estimating unit 103 shown in FIG. 6, the autocovariance function estimating unit 303 estimates the autocovariance function of the body motion signal included in the band signal 1N, and supplies a result of the estimation to the linear prediction filter 304.

Like the linear prediction filter 104 shown in FIG. 6, the linear prediction filter 304 generates the AR model for a body motion signal by using the estimated autocovariance function, and extracts the body motion signal from the band signal 1N in accordance with a result of the body motion signal prediction. The linear prediction filter 304 supplies the extracted body motion signal to the arithmetic unit 306.

The BPF 305 extracts the components in a frequency band W from the measurement signal 1, and supplies a signal containing the extracted components in the frequency band W (this signal will be hereinafter referred to as the band signal 1W) to the arithmetic unit 306 and the selecting unit 309.

The arithmetic unit 306 adds the inverted signal of the body motion signal to the band signal W1, to calculate the difference between the band signal W1 and the body motion signal. The arithmetic unit 306 supplies the selecting unit 309 with a pulse wave signal that is the differential signal between the band signal W1 and the body motion signal.

The combined vector calculating unit 307 calculates a combined vector of the band signal 1N and the band signal 2N. The combined vector calculating unit 307 supplies a result of the combined vector calculation to the determining unit 308.

In accordance with the result of the combined vector calculation, the determining unit 308 determines whether hindrance body motion has been generated. The determining unit 308 supplies the selecting unit 309 with a result of the determination as to generation of hindrance body motion.

In accordance with the result of the determination as to generation of hindrance body motion, the selecting unit 309 selects the pulse wave signal or the band signal 1W as the signal to be used in pulse measurement (this signal will be hereinafter referred to as the pulse measurement signal). The selecting unit 309 supplies the selected pulse measurement signal to the DFT unit 310.

Like the DFT units 107a and 107b shown in FIG. 6, the DFT unit 310 performs DFT on the pulse measurement signal, and supplies a result of frequency analysis of the pulse measurement signal to the band limiting unit 311.

In accordance with the detected value of the previous pulse wave frequency stored in the storage unit 314, the band limiting unit 311 limits the frequency band in which the peak frequency is to be detected. The band limiting unit 311 supplies the peak detecting unit 312 with a result of the frequency analysis of the pulse measurement signal and information indicating the frequency band in which the peak frequency is to be detected.

Like the peak detecting units 108a and 108b shown in FIG. 6, the peak detecting unit 312 detects the peak frequency of the pulse measurement signal. This peak frequency serves as the measured value of the pulse wave frequency. The peak detecting unit 312 supplies the measured value of the pulse wave frequency to the calculating unit 313, and stores the measured value of the pulse wave frequency into the storage unit 314.

In accordance with the pulse wave frequency, the calculating unit 313 calculates the pulse rate. The calculating unit 313 outputs the calculated pulse rate as a measurement result to the outside.

The storage unit 314 stores the measured values of the past pulse wave frequencies.

### [Second Embodiment of a Pulse Measurement Process]

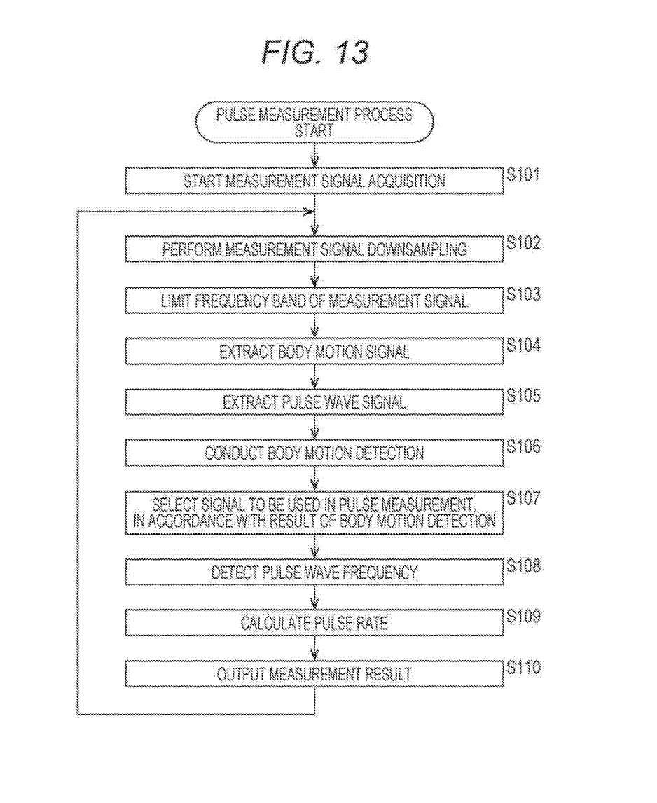

Referring now to the flowchart shown in FIG. 13, a second embodiment of a pulse measurement process to be performed by the measurement device 1 is described.

In step S101, measurement signal acquisition is started, as in the procedure in step S1 in FIG. 8.

In step S102, the decimation filters 301a and 301b perform measurement signal downsampling. Specifically, the decimation filter 301a downsamples the measurement signal 1 at a predetermined rate, and supplies the measurement signal 1 after the downsampling to the BPF 302a and the BPF 305. The decimation filter 301b downsamples the measurement signal 2 at a predetermined rate, and supplies the measurement signal 2 after the downsampling to the BPF 302b.

In step S103, the BPFs 302a and 302b, and the BPF 305 limit the frequency band of the measurement signals. Specifically, the BPF 302a extracts the components in the frequency band N from the measurement signal 1 after the downsampling. The BPF 302a supplies the band signal 1N containing the extracted components in the frequency band N to the autocovariance function estimating unit 303, the linear prediction filter 304, and the combined vector calculating unit 307.

The BPF 302b extracts the components in the frequency band N from the measurement signal 2 after the downsampling. The BPF 302b supplies the band signal 2N containing the extracted components in the frequency band N to the combined vector calculating unit 307.

The BPF 305 extracts the components in the frequency band W from the measurement signal 1 after the downsampling. The BPF 305 supplies the band signal 1W containing the extracted components in the frequency band W to the arithmetic unit 306 and the selecting unit 309.

In step S104, a body motion signal is extracted from the band signal 1N through the same procedure as step S4 in FIG. 8. The extracted body motion signal is supplied to the arithmetic unit 306.

In step S105, a difference between the band signal 1W and the body motion signal is calculated, and a pulse wave signal is extracted through the same procedure as step S5 in FIG. 8. The extracted pulse wave signal is supplied to the selecting unit 309.

In step S106, the body motion detecting unit 333 conducts body motion detection. First, the combined vector calculating unit 307 calculates a combined vector of the band signal 1N and the band signal 2N. The combined vector is a vector having components that are the sampled value (amplitude value) of the band signal 1N and the sampled value (amplitude value) of the band signal 2N at the same sampling time. The combined vector calculating unit 307 supplies a result of the combined vector calculation to the determining unit 308.

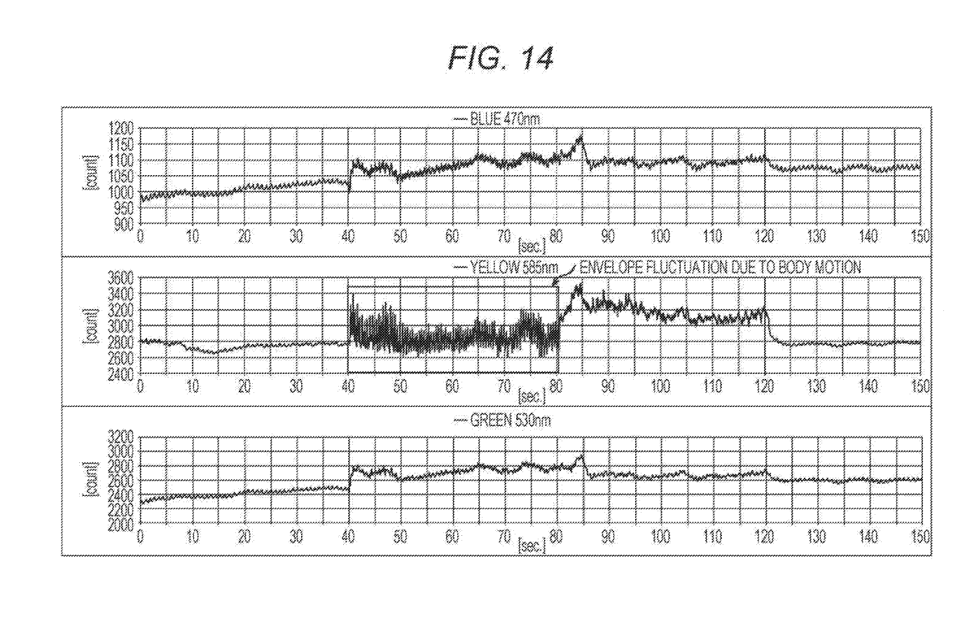

In accordance with the combined vector, the determining unit 308 determines whether hindrance body motion has been generated. Referring now to FIGS. 14 through 16, a method of determining whether hindrance body motion has been generated is described.

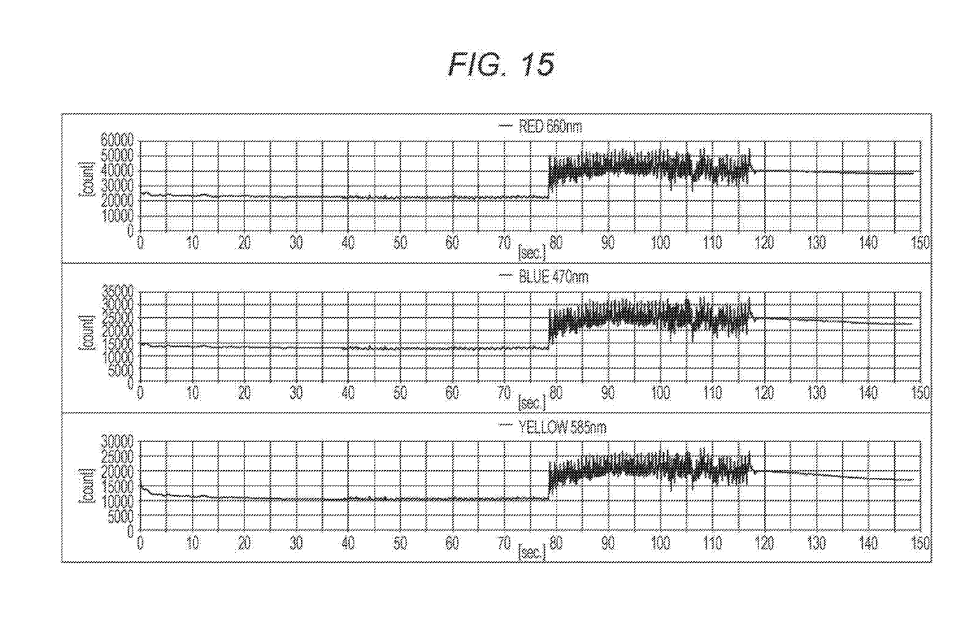

FIGS. 14 and 15 are graphs showing examples of time-series variations of the signal levels of measurement signals. In each graph, the abscissa axis indicates time, and the ordinate axis indicates measurement signal value.

FIG. 14 shows time-series variations of the signal levels of measurement signals in a case where the subject moves only a finger of the arm having the measurement device 1 attached thereto during the period from approximately 40 seconds to approximately 80 seconds, and stays still during the other periods. The uppermost graph shows an example case where blue measurement light of 470 nm in wavelength is used. The second graph from the top shows an example case where yellow measurement light of 585 nm in wavelength is used. The third graph from the top shows an example case where green measurement light of 530 nm in wavelength is used.

Before the subject moves the finger, the measurement signals with respect to the measurement light of all the wavelengths hardly fluctuate but are stable.

When the subject moves the finger after that, the magnitude of fluctuation of the measurement signal with respect to the yellow measurement light becomes conspicuously greater. However, the measurement signals with respect to the measurement light of the other colors increase a little in value, but the magnitude of fluctuation thereof hardly change.

After the subject stops moving the finger, the magnitude of fluctuation of the measurement signal with respect to the yellow measurement light becomes smaller, but the magnitude of fluctuation of the measurement light with respect to the yellow measurement light remains greater than the magnitudes of fluctuation of the measurement signals with respect to the measurement light of the other colors for a while. Also, for a certain time, the values of the measurement signals with respect to the measurement light of all the wavelengths remain greater than those during rest.

After that, the measurement signals with respect to the measurement light of all the wavelengths return to the same stable state as that prior to the movement of the finger.

FIG. 15 shows time-series variations of the signal levels of measurement signals in a case where the subject moves the entire arm having the measurement device 1 attached thereto during the period from approximately 78 seconds to approximately 118 seconds, and stays still during the other periods. The uppermost graph shows an example case where red measurement light of 660 nm in wavelength is used. The second graph from the top shows an example case where blue measurement light of 470 nm in wavelength is used. The third graph from the top shows an example case where yellow measurement light of 585 nm in wavelength is used.

As in the example shown in FIG. 14, before the subject moves the arm, the measurement signals with respect to the measurement light of all the wavelengths hardly fluctuate but are stable.

When the subject moves the arm after that, the values of the measurement signals with respect to the measurement light of all the wavelengths increase, and the magnitudes of fluctuation become greater.

After the subject stops moving the arm, the magnitudes of fluctuation become smaller, but the values of the measurement signals with respect to the measurement light of all the wavelengths remain high.

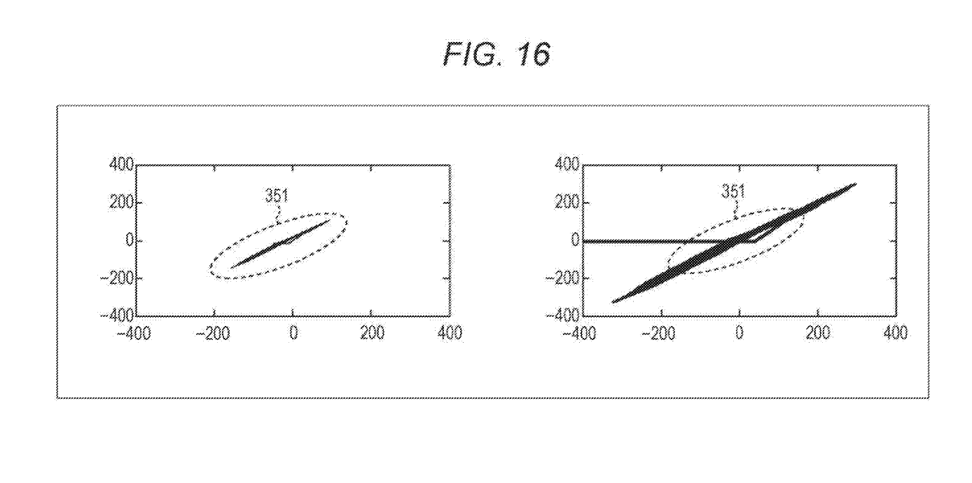

FIG. 16 shows example distributions of a combined vector during one measurement period. The graph on the left side shows an example distribution of a combined vector in a case where the subject stays still. The graph on the right side shows an example distribution of the combined vector in a case where the subject is moving. In both of the graphs on the right side and the left side, the abscissa axis indicates the sample value of the measurement signal (the band signal 1N) with respect to the blue measurement light, and the ordinate axis indicates the sample value of the measurement signal (the band signal 2N) with respect to the yellow measurement light.

As described above with reference to FIGS. 14 and 15, before body motion is generated, the measurement signals with respect to the measurement light of all the wavelengths hardly fluctuate but are stable. Therefore, as shown in the graph on the left side in FIG. 16, the distribution of the combined vector while no hindrance body motion has been generated is expected to fall within a normal zone 351 that is a predetermined range indicated by a dashed line.

When body motion is generated, on the other hand, the value of the measurement signal increases, and the magnitude of fluctuation becomes greater. However, the fluctuations of measurement signals vary depending not only on the types and sizes of body motion but also on the wavelengths of measurement light. That is, as shown in the example in FIG. 14, only the measurement signal with respect to measurement light of a particular wavelength might react sharply depending on the type of body motion.

In view of this, as shown in the graph on the right side in FIG. 16, the distribution of the combined vector in a case where hindrance body motion has been generated varies more widely than the distribution of the combined vector during rest, and spreads out of the normal zone 351. Also, there are cases where the fluctuations of the measurement signals vary with the wavelengths of measurement light, and the combined vector greatly deviates from a positively sloped straight line passing through the origin.

In view of this, when the distribution of the combined vector falls within the normal zone 351, the determining unit 308 determines that no hindrance body motion has been generated. When the distribution of the combined vector does not fall within the normal zone 351, on the other hand, the determining unit 308 determines that hindrance body motion has been generated. The determining unit 308 supplies a determination result to the selecting unit 309.

In step S107, the selecting unit 309 selects the signal (the pulse measurement signal) to be used in pulse measurement, in accordance with a result of the body motion detection. Specifically, when it is determined that hindrance body motion has been generated, the selecting unit 309 selects the pulse wave signal supplied from the arithmetic unit 306 as the pulse measurement signal, and supplies the pulse measurement signal to the DFT unit 310. When it is determined that no hindrance body motion has been generated, on the other hand, the selecting unit 309 selects the band signal 1W supplied from the BPF 305 as the pulse measurement signal, and supplies the pulse measurement signal to the DFT unit 310.

In step S108, the frequency detecting unit 334 detects the pulse wave frequency. Specifically, through the same procedure as step S6 in FIG. 8, the DFT unit 310 carries out frequency analysis of the pulse measurement signal, and supplies a result of the analysis to the band limiting unit 311.

Through the same procedure as step S6 in FIG. 8, the band limiting unit 311 limits the frequency band in which the peak frequency is to be detected, in accordance with the detected value of the previous pulse wave frequency stored in the storage unit 314. The band limiting unit 311 supplies the peak detecting unit 312 with a result of the frequency analysis of the pulse measurement signal and the information indicating the frequency band in which the peak frequency is to be detected.

Through the same procedure as step S6 in FIG. 8, the peak detecting unit 312 detects the peak frequency of the pulse measurement signal. This peak frequency serves as the measured value of the pulse wave frequency.

In view of this, when it is determined that hindrance body motion has been generated, the peak frequency of the pulse wave signal serves as the measured value of the current pulse wave frequency. When it is determined that no hindrance body motion has been generated, on the other hand, the peak frequency of the band signal 1W serves as the measured value of the current pulse wave frequency.

The peak detecting unit 312 supplies the information indicating the pulse wave frequency to the calculating unit 112, and stores the information into the storage unit 113.

In step S109, the pulse rate is calculated as in the procedure in step S10 in FIG. 8.

In step S110, a measurement result is output as in the procedure in step S11 in FIG. 8.

After that, the process returns to step S102, and the procedure in step S102 and the procedures thereafter are repeated.

In the above manner, influence of body motion can be eliminated, and the pulse wave and the pulse of the subject can be accurately measured with a small amount of calculation, as in the first embodiment.

### 4. Third Embodiment

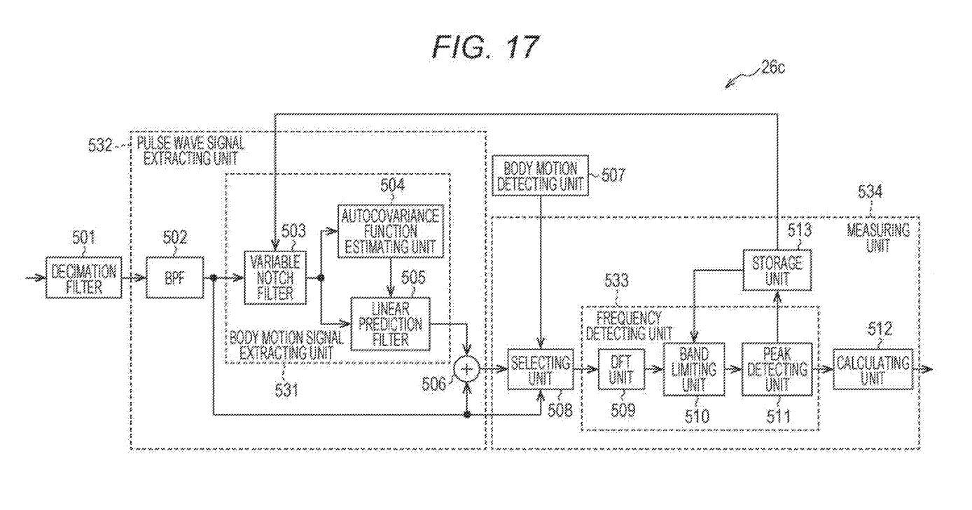

Referring now to FIGS. 17 through 19, a third embodiment of the present technology is described. The second embodiment differs from the first and second embodiments in the body motion signal extraction method and the body motion detection method. In the third embodiment, one kind of wavelength measurement light is used as in the first embodiment, for example.

[Example Structure of an Arithmetic Processing Unit 26c]

In the third embodiment, an arithmetic processing unit 26c shown in FIG. 17, instead of the arithmetic processing unit 26a shown in FIG. 6 and the arithmetic processing unit 26b shown in FIG. 12, is used in a measurement device 1. The arithmetic processing unit 26c is designed to include a decimation filter 501, a bandpass filter (BPF) 502, a variable notch filter 503, an autocovariance function estimating unit 504, a linear prediction filter 505, an arithmetic unit 506, a body motion detecting unit 507, a selecting unit 508, a discrete Fourier transform (DFT) unit 509, a band limiting unit 510, a peak detecting unit 511, a calculating unit 512, and a storage unit 513.

The variable notch filter 503, the autocovariance function estimating unit 504, and the linear prediction filter 505 constitute a body motion signal extracting unit 531. The BPF 502, the arithmetic unit 506, and the body motion signal extracting unit 531 constitute a pulse wave signal extracting unit 532. The DFT unit 509, the band limiting unit 510, and the peak detecting unit 511 constitute a frequency detecting unit 533. The selecting unit 508, the calculating unit 512, the storage unit 513, and the frequency detecting unit 533 constitute a measuring unit 534.

The decimation filter 501 has the same functions as those of the decimation filter 101 shown in FIG. 6. The BPF 502 has the same functions as those of the BPF 105 shown in FIG. 6. The autocovariance function estimating unit 504 has the same functions as those of the autocovariance function estimating unit 103 shown in FIG. 6. The linear prediction filter 505 has the same functions as those of the linear prediction filter 104 shown in FIG. 6. The arithmetic unit 506 has the same functions as those of the arithmetic unit 106 shown in FIG. 6. The selecting unit 508 has the same functions as those of the selecting unit 309 shown in FIG. 12. The DFT unit 509 has the same functions as those of the DFT units 107a and 107b shown in FIG. 6. The band limiting unit 510 has the same functions as those of the band limiting unit 311 shown in FIG. 12. The peak detecting unit 511 has the same functions as those of the peak detecting unit 312 shown in FIG. 12. The calculating unit 512 has the same functions as those of the calculating unit 112 shown in FIG. 6.

The decimation filter 501 performs downsampling on a measurement signal. The decimation filter 501 supplies the measurement signal after the downsampling, to the BPF 502.

The BPF 502 extracts the components in a frequency band W from the measurement signal, and supplies a band signal W containing the extracted components in the frequency band W to the variable notch filter 503, the arithmetic unit 506, and the selecting unit 508.

The variable notch filter 503 is a zero-phase filter having a variable attenuation band. The variable notch filter 503 sets an attenuation band containing the measured value of the previous pulse wave frequency stored in the storage unit 513, and attenuates the components in the attenuation band among the frequency components in the band signal W. The variable notch filter 503 supplies the signal after the attenuation (this signal will be hereinafter referred to as the band signal W) to the autocovariance function estimating unit 504 and the linear prediction filter 505.

Since the variable notch filter 503 is a zero-phase filter, temporal axis information such as body motion components is stored as it is in the band signal W after the attenuation.

Like the autocovariance function estimating unit 103 shown in FIG. 6, the autocovariance function estimating unit 504 estimates the autocovariance function of the body motion signal included in the band signal W, and supplies a result of the estimation to the linear prediction filter 505.

Like the linear prediction filter 104 shown in FIG. 6, the linear prediction filter 505 generates the AR model for a body motion signal by using the estimated autocovariance function, and extracts the body motion signal from the band signal Win accordance with a result of the body motion signal prediction. The linear prediction filter 505 supplies the extracted body motion signal to the arithmetic unit 506.

The arithmetic unit 506 adds the inverted signal of the body motion signal to the band signal W, to calculate the difference between the band signal W and the body motion signal. The arithmetic unit 506 supplies the selecting unit 508 with a pulse wave signal that is the differential signal between the band signal W and the body motion signal.

The body motion detecting unit 507 conducts body motion detection by a predetermined method, and determines whether hindrance body motion has been generated. The body motion detecting unit 507 supplies the selecting unit 508 with a result of the determination as to generation of hindrance body motion.

In accordance with the result of the determination as to generation of hindrance body motion, the selecting unit 508 selects the pulse wave signal or the band signal W as the pulse measurement signal. The selecting unit 508 supplies the selected pulse measurement signal to the DFT unit 509.

Like the DFT units 107a and 107b shown in FIG. 6, the DFT unit 509 performs DFT on the pulse measurement signal, and supplies a result of frequency analysis of the pulse measurement signal to the band limiting unit 510.

In accordance with the detected value of the previous pulse wave frequency stored in the storage unit 513, the band limiting unit 510 limits the frequency band in which the peak frequency is to be detected. The band limiting unit 510 supplies the peak detecting unit 511 with a result of the frequency analysis of the pulse measurement signal and the information indicating the frequency band in which the peak frequency is to be detected.

Like the peak detecting units 108a and 108b shown in FIG. 6, the peak detecting unit 511 detects the peak frequency of the pulse measurement signal. This peak frequency serves as the measured value of the pulse wave frequency. The peak detecting unit 511 supplies the measured value of the pulse wave frequency to the calculating unit 512, and stores the measured value of the pulse wave frequency into the storage unit 513.

In accordance with the pulse wave frequency, the calculating unit 512 calculates the pulse rate. The calculating unit 512 outputs the calculated pulse rate as a measurement result to the outside.

The storage unit 513 stores the measured values of the past pulse wave frequencies.

### [Third Embodiment of a Pulse Measurement Process]

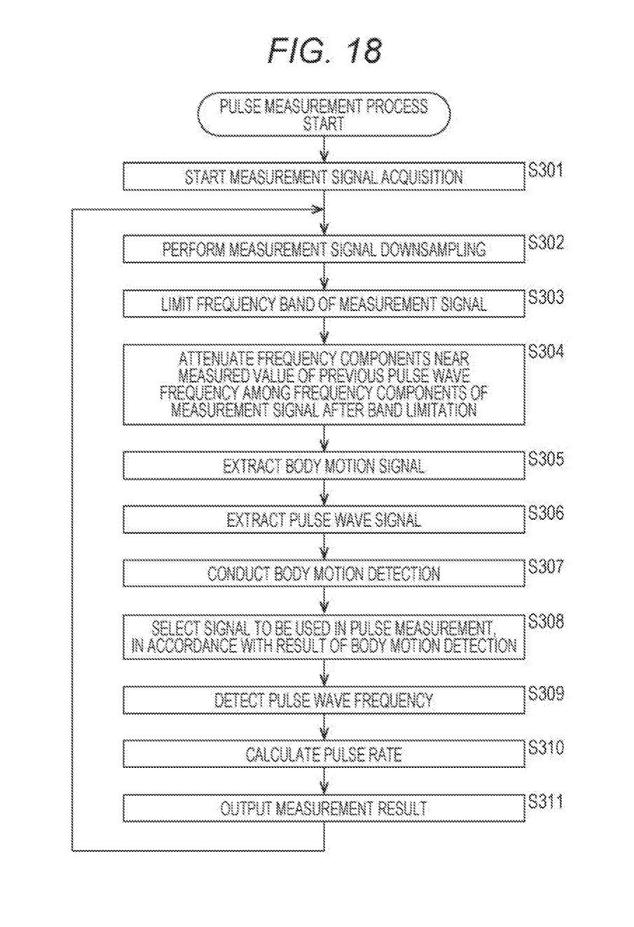

Referring now to the flowchart shown in FIG. 18, a third embodiment of a pulse measurement process to be performed by the measurement device 1 is described.

In step S301, measurement signal acquisition is started, as in the procedure in step S1 in FIG. 8.

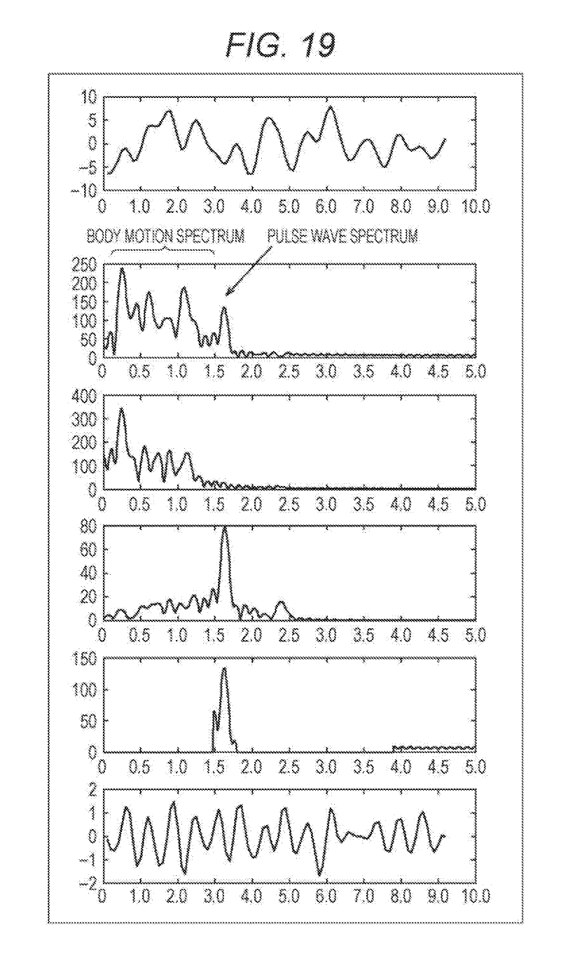

The uppermost graph in FIG. 19 shows an example of the waveform of a measurement signal. In this graph, the abscissa axis indicates time, and the ordinate axis indicates amplitude value.

In step S302, measurement signal downsampling is performed as in the procedure in step S2 in FIG. 8. The measurement signal after the downsampling is supplied to the BPF 502.

In step S303, the BPF 502 limits the frequency band of the measurement signal. Specifically, the BPF 502 extracts the components in the frequency band W from the measurement signal after the downsampling. The BPF 502 supplies the band signal W containing the extracted components in the frequency band W to the variable notch filter 503, the arithmetic unit 506, and the selecting unit 508.

The second graph from the top in FIG. 19 shows an example of the frequency distribution of the band signal W after the frequency band of the measurement signal shown in the uppermost graph is limited. In this graph, the abscissa axis indicates frequency, and the ordinate axis indicates spectrum intensity. In this example, a pulse wave spectrum appears at approximately 1.6 Hz, and a body motion component spectrum appears at 1.5 Hz and lower. There exist few frequency components at 1.8 Hz and higher.

In step S304, the variable notch filter 503 attenuates the frequency components in the vicinity of the measured value of the previous pulse wave frequency among the frequency components of the measurement signal (the band signal W) after the band limitation. Specifically, the variable notch filter 503 reads the measured value of the previous pulse wave frequency from the storage unit 513. The variable notch filter 503 sets an attenuation band that is a predetermined range containing the measured value of the previous pulse wave frequency, for example. A predetermined band having the measured value of the previous pulse wave frequency at its center is set as the attenuation band, for example.

The variable notch filter 503 attenuates the components in the set attenuation band among the frequency components of the band signal W. The variable notch filter 503 supplies the band signal W after the attenuation to the autocovariance function estimating unit 504 and the linear prediction filter 505.

Since the pulse wave frequency does not rapidly vary, most pulse wave components are expected to be included in the frequency components attenuated by the variable notch filter 503. As a result, few pulse wave components are included in the band signal W.

In step S305, a body motion signal is extracted from the band signal W through the same procedure as step S4 in FIG. 8. The extracted body motion signal is supplied to the arithmetic unit 506.

As described above, the band signal W include few pulse wave components. As a result, few pulse wave components are included in the body motion signal, regardless of the amount of the body motion components included in the measurement signal. This aspect differs from the above described embodiments.

The third graph from the top in FIG. 19 shows an example of the frequency distribution of the body motion signal extracted from the band signal W shown in the second graph from the top. In this graph, the abscissa axis indicates frequency, and the ordinate axis indicates spectrum intensity. As shown in this graph, the pulse wave spectrum included in the band signal W is lost in the body motion signal.

In step S306, a difference between the band signal W and the body motion signal is calculated, and a pulse wave signal is extracted through the same procedure as step S5 in FIG. 8. The extracted pulse wave signal is supplied to the selecting unit 508.

Since few pulse wave components are included in the body motion signal, as described above, the pulse wave components are certainly included in the pulse wave signal.

The fourth graph from the top in FIG. 19 shows an example of the frequency components of the pulse wave signal obtained by calculating the difference between the band signal W shown in the second graph from the top and the body motion signal shown in the third graph from the top. In this graph, the abscissa axis indicates frequency, and the ordinate axis indicates spectrum intensity. As shown in this graph, the pulse wave components are extracted from the band signal W, and the components at the other frequencies are attenuated. As a result, the pulse wave components become much greater than the body motion components.

In step S307, the body motion detecting unit 507 conducts body motion detection. For example, the body motion detecting unit 507 conducts body motion detection in accordance with a detection signal supplied from a sensor (not shown) in the measurement device 1, such as a triaxial acceleration sensor or a gyro sensor. In accordance with a result of the body motion detection, the body motion detecting unit 507 determines whether hindrance body motion has been generated. The body motion detecting unit 507 supplies the selecting unit 508 with a result of the determination as to generation of hindrance body motion.

In step S308, the signal (the pulse measurement signal) to be used in pulse measurement is selected in accordance with the result of the body motion detection, as in the procedure in step S107 in FIG. 13.

In step S309, the pulse wave frequency is detected as in the procedure in step S108 in FIG. 13.

The fifth graph from the top in FIG. 19 shows the frequency distribution of the pulse measurement signal after the detection range is limited in the procedure in step S309 in a case where the pulse wave signal shown in the fourth graph from the top is selected as the pulse measurement signal. In this graph, the abscissa axis indicates frequency, and the ordinate axis indicates spectrum distribution.

The lowermost graph in FIG. 19 shows the waveform of the pulse wave signal after the body motion signal having the frequency distribution shown in the third graph from the top is subtracted from the measurement signal shown in the uppermost graph. In this graph, the abscissa axis indicates time, and the ordinate axis indicates amplitude value.

In step S310, the pulse rate is calculated as in the procedure in step S109 in FIG. 13.

In step S311, a measurement result is output as in the procedure in step S110 in FIG. 13.

After that, the process returns to step S302, and the procedure in step S302 and the procedures thereafter are repeated.

In the above manner, influence of body motion can be eliminated, and the pulse wave and the pulse of the subject can be accurately measured with a small amount of calculation, as in the first and second embodiments.

In the third embodiment, the number of BPFs can be made smaller than that in the first and second embodiments by one. Further, in the third embodiment, a pulse wave signal containing pulse wave components extracted with high precision can be generated.

### 5. Modifications

The following is a description of modifications of the above described embodiments of the present technology.

### [Modifications Relating to Body Motion Detection Methods]

The above described body motion detection method is an example, and some other methods may be used.

For example, in a case where body motion has been generated, the signal level of a measurement signal varies greatly, as described above with reference to FIGS. 14 and 15. In view of this, the body motion detecting unit may detect body motion in accordance with a change in the signal level (the amplitude value or the magnitude of amplitude, for example) of a measurement signal, for example. When the amount of change in the signal level of a measurement signal becomes equal to or greater than a predetermined threshold value, for example, the body motion detecting unit 133 or the body motion detecting unit 333 may determine that hindrance body motion has been generated.

Also, the fluctuation of a measurement signal becomes greater depending on the wavelength of the measurement light and the type of body motion, as described above with reference to FIGS. 14 and 15. In a case where measurement light of two or more wavelengths is used, for example, the body motion detecting unit may determine that hindrance body motion has been generated, when at least one of the measurement signals corresponding to the respective wavelengths of the measurement light satisfies a predetermined condition.

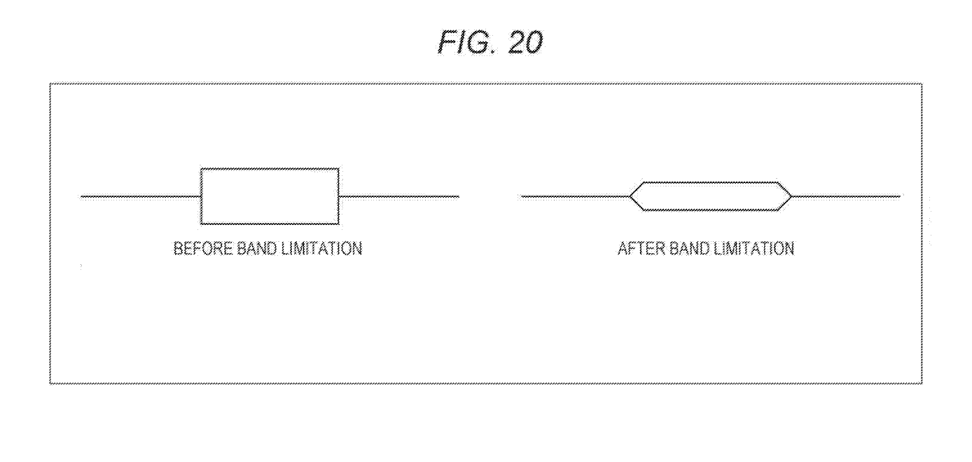

FIG. 20 schematically shows the envelope of a measurement signal before the frequency band is limited, and the envelope of the measurement signal after the limitation. As shown in this drawing, after the band limitation, rises and falls of the fluctuation of the measurement signal become slower. As a result, there may be delays in body motion detection, or body motion may be wrongly detected. To counter this, the body motion detecting unit may remove the direct-current components, and then conduct body motion detection in accordance with a measurement signal not subjected to frequency band limitation and downsampling, for example.

Body motion detection may be conducted by a combination of two or more body motion detection methods.

### [Modifications Relating to Pulse Wave Frequency Detection Methods]

In the examples described above, the peak frequency of the pulse wave signal or the band signal W (or the band signal 1W) is selected as the pulse wave frequency in accordance with a result of body motion detection or the like. However, a pulse wave frequency may be measured by some other methods.

For example, in a case where a peak with a spectrum intensity equal to or higher than a predetermined threshold value exists within a predetermined band having the measured value of the previous pulse wave frequency at its center in the frequency distribution of a measurement signal, the frequency corresponding to the peak may be measured as the pulse wave frequency.

In the third embodiment, pulse wave components are certainly included in a pulse wave signal. In view of this, the body motion detection process may be skipped, and the peak frequency of the pulse wave signal may be constantly measured as the pulse wave frequency, for example.

### [Modifications Relating to AR Models for Body Motion Signals]

The order of the AR model for the linear prediction filter may be changed in accordance with the level (the amplitude value, for example) of a measurement signal (the band signal W or the band signal 1W), for example.

Specifically, in a case where the ratio of body motion components to pulse wave components is high, the precision of separation between the body motion components and the pulse wave components by the linear prediction filter hardly becomes lower, even if the order of the AR model for the linear prediction filter is made higher. As the order of the AR model for the linear prediction filter is made higher, the precision of noise separation by the linear prediction filter also becomes higher. In a case where the ratio of body motion components to pulse wave components is low, on the other hand, the precision of separation between the body motion components and the pulse wave components by the linear prediction filter becomes lower, if the order of the AR model for the linear prediction filter is made higher.

Also, as the level of a measurement signal becomes higher, the body motion components included in the measurement signal become greater, and the ratio of the body motion components to the pulse wave components becomes higher.

In view of this, the linear prediction filter may increase the order of the AR model as the level of a measurement signal becomes higher, and lower the order of the AR model as the level of the measurement signal becomes lower, for example.

Also, the AR model for a body motion signal may be generated by a method other than the Yule-Walker's method.

### [Modifications Relating to Downsampling]

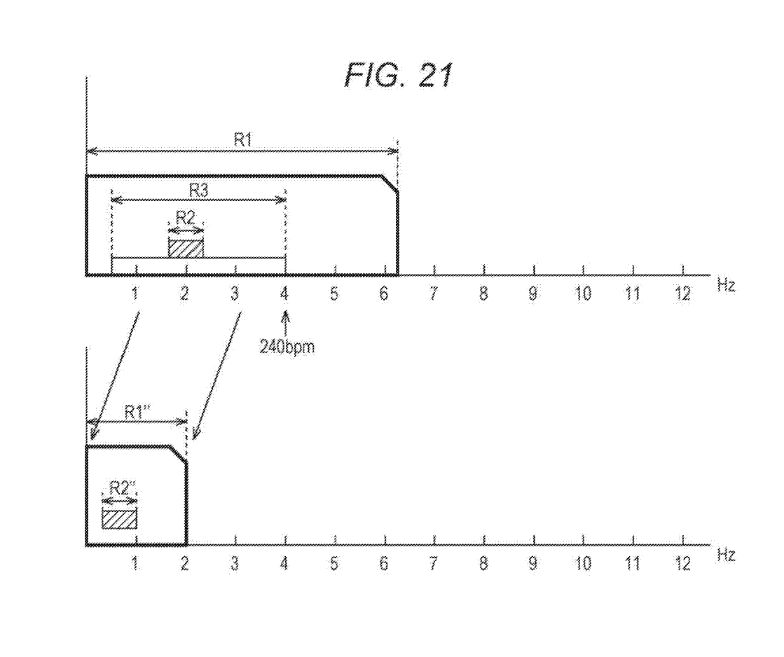

The components in a predetermined band (one to two octaves, for example) having the previous pulse wave frequency at its center are extracted by a BPF, and the frequency of the extracted signal is made to shift, so that the downsampling rate for a measurement signal can be further lowered, for example. Referring now to FIG. 21, this specific example is described. The upper graph in FIG. 21 is the same as the graph in FIG. 9.

First, the components in the frequency band in a detection range R2 are extracted from a measurement signal by a BPF, for example. The frequency band of the extracted signal is then made to shift to the frequency band R2″ shown in the lower graph. With this, pulse wave frequency detection becomes possible, if the frequency components that can be detected from the measurement signal after downsampling can be secured at least in a range R1″ that is narrower than the range R1. Thus, the downsampling rate for the measurement signal can be further lowered.

Alternatively, downsampling may not be performed, and the sampling frequency of a measurement signal may be lowered within such a range that the pulse wave frequency can be measured, for example.

### [Modifications Relating to Measurement Results]

In the examples described above, a pulse rate is output as a measurement result. However, a pulse wave frequency may be output as a measurement result, for example.

Also, a pulse wave signal may be output as a measurement result, for example. Alternatively, the pulse wave signal or the band signal W (or the band signal 1W) is selected in accordance with a result of body motion detection, and the selected signal may be output as a result of pulse wave measurement. In any of these cases, it is preferable to remove noise before a signal is output.

Further, two or more signals among the pulse rate, the pulse wave frequency, and the pulse wave signal (or the band signal W or the band signal 1W) may be output as a measurement result.

Also, a body motion signal may be included in a measurement result, for example.

### [Other Modifications]

The various numerical values (such as the frequencies, the wavelengths, the numbers of samples, and the downsampling rates) mentioned in the above description are merely examples, and numerical values other than the above may also be used.

Instead of the measuring unit 135 in FIG. 6, the measuring unit 335 in FIG. 12 or the measuring unit 534 in FIG. 17 may be used. Conversely, instead of the measuring unit 335 in FIG. 12 or the measuring unit 534 in FIG. 17, the measuring unit 135 in FIG. 6 may be used.

Also, the fluctuation of the waveform of a measurement signal varies depending on the wavelength of the measurement light and the type of body motion, as described above with reference to FIGS. 14 and 15. Body motion may be categorized into several types, in accordance with the differences in fluctuation of the waveform, for example.

Further, the sequences of the procedures in steps in each of the flowcharts in FIGS. 8, 13, and 18 may be changed as appropriate, or these procedures may be carried out in parallel.

The measurement device 1 may be formed with a system including more than one device. For example, part of or all of the arithmetic processing unit 26 may be provided in a different device from the device to be attached to the subject, and measurement signals may be exchanged between the devices through wireless communication or cable communication.

In the examples described above, the measurement device is attached to an arm of a person. However, the present technology can also be applied to any measurement device to be attached to a portion other than an arm. Also, the measurement device may have a shape other than the above described shape of a wristband.

The present technology can also be applied in cases where the pulse wave and the pulse of a living creature other than a human being are to be measured.

### [Example Structure of a Computer]

The above described series of processes can be performed by hardware, and can also be performed by software. When the series of processes are to be performed by software, the program that forms the software is installed into a computer. Here, the computer may be a computer incorporated into special-purpose hardware, or may be a general-purpose personal computer that can execute various kinds of functions as various kinds of programs are installed thereinto.

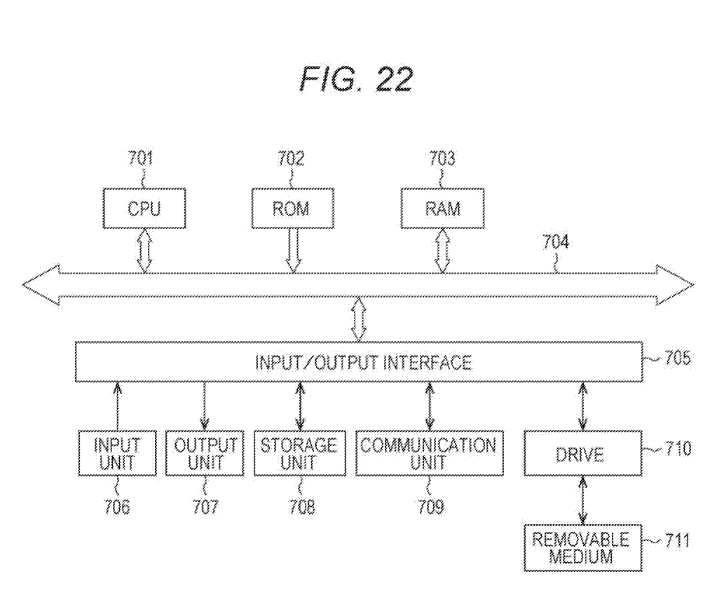

FIG. 22 is a block diagram showing an example configuration of the hardware of a computer that performs the above described series of processes in accordance with a program.

In the computer, a CPU (Central Processing Unit) 701, a ROM (Read Only Memory) 702, and a RAM (Random Access Memory) 703 are connected to one another by a bus 704.

An input/output interface 705 is further connected to the bus 704. An input unit 706, an output unit 707, a storage unit 708, a communication unit 709, and a drive 710 are connected to the input/output interface 705.

The input unit 706 is formed with a keyboard, a mouse, a microphone, and the like. The output unit 707 is formed with a display, a speaker, and the like. The storage unit 708 is formed with a hard disk, a nonvolatile memory, or the like. The communication unit 709 is formed with a network interface or the like. The drive 710 drives a removable medium 711 that is a magnetic disk, an optical disk, a magneto optical disk, a semiconductor memory, or the like.

In the computer having the above described structure, the CPU 701 loads a program stored in the storage unit 708 into the RAM 703 via the input/output interface 705 and the bus 704, for example, and executes the program, so that the above described series of processes are performed.

The program to be executed by the computer (the CPU 701) may be recorded on the removable medium 711 as a packaged medium to be provided, for example. Alternatively, the program can be provided via a wired or wireless transmission medium such as a local area network, the Internet, or digital satellite broadcasting.

In the computer, the program can be installed into the storage unit 708 via the input/output interface 705 when the removable medium 711 is mounted on the drive 710. The program can also be received by the communication unit 709 via a wired or wireless transmission medium, and be installed into the storage unit 708. Also, the program may be installed beforehand into the ROM 702 or the storage unit 708.

The program to be executed by the computer may be a program for performing processes in chronological order in accordance with the sequence described in this specification, or may be a program for performing processes in parallel or performing a process when necessary, such as when there is a call.

In this specification, a system means an assembly of components (apparatuses, modules (parts), and the like), and not all the components need to be provided in the same housing. In view of this, devices that are housed in different housings and are connected to each other via a network form a system, and one device having modules housed in one housing is also a system.

Further, it should be noted that embodiments of the present technology are not limited to the above described embodiments, and various modifications may be made to them without departing from the scope of the present technology.

For example, the present technology can be embodied in a cloud computing structure in which one function is shared among devices via a network, and processing is performed by the devices cooperating with one another.

The respective steps described with reference to the above described flowcharts can be carried out by one device or can be shared among devices.

In a case where more than one process is included in one step, the processes included in the step can be performed by one device or can be shared among devices.

The advantageous effects described in this specification are merely examples, and the advantageous effects of the present technology are not limited to them and may include other effects.

Further, it should be noted that embodiments of the present technology are not limited to the above described embodiments, and various modifications may be made to them without departing from the scope of the present technology.

The present technology can also be in the following forms, for example.

(1) A measurement device including:

a body motion signal extracting unit that extracts a body motion signal containing a component generated by body motion from a first band signal containing the components in a first frequency band of a first measurement signal acquired by illuminating a portion having a pulse with light of a first wavelength; and

an arithmetic unit that generates a pulse wave signal that is a differential signal between a second band signal and the body motion signal, the second band signal containing the components in a second frequency band of the first measurement signal.

(2) The measurement device of (1), wherein

the body motion signal extracting unit predicts and extracts the body motion signal, using an autoregressive model.

(3) The measurement device of (2), wherein

the body motion signal extracting unit generates the autoregressive model within a range of the fifth through twelfth orders, using the Yule-Walker's method.

(4) The measurement device of (3), wherein

the body motion signal extracting unit sets the order of the autoregressive model in accordance with the level of the first band signal.

(5) The measurement device of any of (1) through (4), further including:

a body motion detecting unit that detects the body motion; and

a measuring unit that measures a pulse wave frequency in accordance with the pulse wave signal or the second band signal, whichever is selected in accordance with a result of the detection of the body motion.

(6) The measurement device of (5), wherein

the body motion signal extracting unit extracts the body motion signal from a signal having attenuated frequency components in a band containing the measured value of the previous pulse wave frequency, the attenuated frequency components being of the frequency components of the first band signal.

(7) The measurement device of (5), wherein

the measuring unit includes:

a frequency detecting unit that detects a first peak frequency that is the peak frequency of the pulse wave signal, and a second peak frequency that is the peak frequency of the second band signal; and

a selecting unit that selects the pulse wave frequency in accordance with at least one of the result of the detection of the body motion and the measured value of the previous pulse wave frequency, the pulse wave frequency being the first peak frequency or the second peak frequency.

(8) The measurement device of (7), wherein

the frequency detecting unit limits the frequency band in which the first peak frequency and the second peak frequency are to be detected, in accordance with the measured value of the previous pulse wave frequency.

(9) The measurement device of (7) or (8), wherein

the frequency detecting unit detects the first peak frequency in accordance with a result of Fourier transform of the pulse wave signal subjected to padding with a sample of the value “0”, and detects the second peak frequency in accordance with a result of Fourier transform of the second band signal subjected to padding with a sample of the value “0”.

(10) The measurement device of (5), wherein

the measuring unit includes:

a selecting unit that selects the pulse wave signal or the second band signal in accordance with the result of the detection of the body motion; and

a frequency detecting unit that detects the pulse wave frequency that is the peak frequency of the signal selected by the selecting unit.

(11) The measurement device of (10), wherein

the frequency detecting unit limits the frequency band in which the peak frequency is to be detected, in accordance with the measured value of the previous pulse wave frequency.

(12) The measurement device of (10) or (11), wherein

the frequency detecting unit detects the peak frequency in accordance with a result of Fourier transform of a signal obtained by performing padding on the signal selected by the selecting unit with a sample of the value “0”.

(13) The measurement device of any of (5) through (12), wherein

the body motion detecting unit detects the body motion in accordance with the frequency distribution of the body motion signal.

(14) The measurement device of any of (5) through (13), wherein

the body motion detecting unit detects the body motion in accordance with the distribution of a combined vector of a third band signal and the first band signal, the third band signal containing the components in the first frequency band of a second measurement signal acquired by illuminating the portion having the pulse with light of a second wavelength.

(15) The measurement device of any of (5) through (14), wherein

the body motion detecting unit detects the body motion in accordance with fluctuation of the first measurement signal and fluctuation of a second measurement signal acquired by illuminating the portion having the pulse with light of a second wavelength.

(16) The measurement device of any of (5) through (15), wherein the measuring unit calculates a pulse rate in accordance with the pulse wave frequency.

(17) The measurement device of any of (1) through (16), further including:

a first filter that extracts the first band signal from the first measurement signal; and

a second filter that extracts the second band signal from the first measurement signal, wherein

the second frequency band includes the range of pulse wave frequencies to be measured, and the largest value in the second frequency band is larger than the largest value in the first frequency band.

(18) The measurement device of any of (1) through (16), further including

a filter that extracts the first band signal from the first measurement signal, wherein:

the first frequency band is the same as the second frequency band and includes a range of pulse wave frequencies to be measured; and

the first band signal is the same as the second band signal.

(19) A measurement method including:

a body motion signal extraction step of extracting a body motion signal containing a component generated by body motion from a first band signal containing the components in a first frequency band of a first measurement signal acquired by illuminating a portion having a pulse with light of a predetermined wavelength; and

an arithmetic step of generating a pulse wave signal that is a differential signal between a second band signal and the body motion signal, the second band signal containing the components in a second frequency band of the first measurement signal.

### REFERENCE SIGNS LIST

1 Measurement device
11 Main unit
22, 22a-22c LED
23 Light receiving IC
26, 26a-26c Arithmetic processing unit
51 LED driver
53 Light receiving element
54 AD converter
101 Decimation filter
102 BPF
103 Autocovariance function estimating unit
104 Linear prediction filter
105 BPF
106 Arithmetic unit
107a, 107b DFT unit
108a, 108b Peak detecting unit
109 DFT unit
110 Determining unit
111 Selecting unit
112 Calculating unit
131 Body motion signal extracting unit
132 Pulse wave signal extracting unit
133 Body motion detecting unit
134 Frequency detecting unit
135 Measuring unit
301a, 301b Decimation filter
302a, 302b BPF
303 Autocovariance function estimating unit
304 Linear prediction filter
305 BPF
306 Arithmetic unit
307 Combined vector generating unit
308 Determining unit
309 Selecting unit
310 DFT unit
311 Band limiting unit
312 Peak detecting unit
313 Calculating unit
331 Body motion signal extracting unit
332 Pulse wave signal extracting unit
333 Body motion detecting unit
334 Frequency detecting unit
335 Measuring unit
501 Decimation filter
502 BPF
503 Variable notch filter
504 Autocovariance function estimating unit
505 Linear prediction filter
506 Arithmetic unit
507 Body motion detecting unit
508 Determining unit
509 DFT unit
510 Band limiting unit
511 Peak detecting unit
512 Calculating unit
531 Body motion signal extracting unit
532 Pulse wave signal extracting unit
533 Frequency detecting unit
534 Measuring unit

1. A measurement device comprising:
a body motion signal extracting unit configured to extract a body motion signal containing a component generated by body motion from a first band signal containing a component in a first frequency band of a first measurement signal acquired by illuminating a portion having a pulse with light of a first wavelength; and
an arithmetic unit configured to generate a pulse wave signal, the pulse wave signal being a differential signal between a second band signal and the body motion signal, the second band signal containing a component in a second frequency band of the first measurement signal.

2. The measurement device according to claim 1, wherein
the body motion signal extracting unit predicts and extracts the body motion signal, using an autoregressive model.

3. The measurement device according to claim 2, wherein
the body motion signal extracting unit generates the autoregressive model within a range of the fifth through twelfth orders, using a Yule-Walker's method.

4. The measurement device according to claim 3, wherein
the body motion signal extracting unit sets the order of the autoregressive model in accordance with the level of the first band signal.

5. The measurement device according to claim 1, further comprising:
a body motion detecting unit configured to detect the body motion; and
a measuring unit configured to measure a pulse wave frequency in accordance with one of the pulse wave signal and the second band signal, the one of the pulse wave signal and the second band signal being selected in accordance with a result of the detection of the body motion.

6. The measurement device according to claim 5, wherein
the body motion signal extracting unit extracts the body motion signal from a signal having an attenuated frequency component in a band containing a measured value of a previous pulse wave frequency, the attenuated frequency component being of frequency components of the first band signal.

7. The measurement device according to claim 5, wherein
the measuring unit includes:
a frequency detecting unit configured to detect a first peak frequency and a second peak frequency, the first peak frequency being a peak frequency of the pulse wave signal, the second peak frequency being a peak frequency of the second band signal; and
a selecting unit configured to select the pulse wave frequency in accordance with at least one of the result of the detection of the body motion and a measured value of a previous pulse wave frequency, the pulse wave frequency being one of the first peak frequency and the second peak frequency.

8. The measurement device according to claim 7, wherein
the frequency detecting unit limits a frequency band in which the first peak frequency and the second peak frequency are to be detected, in accordance with the measured value of the previous pulse wave frequency.

9. The measurement device according to claim 7, wherein
the frequency detecting unit detects the first peak frequency in accordance with a result of Fourier transform of the pulse wave signal subjected to padding with a sample of the value “0”, and detects the second peak frequency in accordance with a result of Fourier transform of the second band signal subjected to padding with a sample of the value “0”.

10. The measurement device according to claim 5, wherein the measuring unit includes:
a selecting unit configured to select one of the pulse wave signal and the second band signal in accordance with the result of the detection of the body motion; and
a frequency detecting unit configured to detect the pulse wave frequency, the pulse wave frequency being a peak frequency of the signal selected by the selecting unit.

11. The measurement device according to claim 10, wherein
the frequency detecting unit limits a frequency band in which the peak frequency is to be detected, in accordance with a measured value of a previous pulse wave frequency.

12. The measurement device according to claim 10, wherein
the frequency detecting unit detects the peak frequency in accordance with a result of Fourier transform of a signal obtained by performing padding on the signal selected by the selecting unit with a sample of the value “0”.

13. The measurement device according to claim 5, wherein
the body motion detecting unit detects the body motion in accordance with a frequency distribution of the body motion signal.

14. The measurement device according to claim 5, wherein
the body motion detecting unit detects the body motion in accordance with a distribution of a combined vector of a third band signal and the first band signal, the third band signal containing a component in the first frequency band of a second measurement signal acquired by illuminating the portion having the pulse with light of a second wavelength.

15. The measurement device according to claim 5, wherein
the body motion detecting unit detects the body motion in accordance with fluctuation of the first measurement signal and fluctuation of a second measurement signal acquired by illuminating the portion having the pulse with light of a second wavelength.

16. The measurement device according to claim 5, wherein
the measuring unit calculates a pulse rate in accordance with the pulse wave frequency.

17. The measurement device according to claim 1, further comprising:
a first filter configured to extract the first band signal from the first measurement signal; and
a second filter configured to extract the second band signal from the first measurement signal, wherein
the second frequency band includes a range of pulse wave frequencies to be measured, and the largest value in the second frequency band is larger than the largest value in the first frequency band.

18. The measurement device according to claim 1, further comprising
a filter configured to extract the first band signal from the first measurement signal, wherein:
the first frequency band is the same as the second frequency band and includes a range of pulse wave frequencies to be measured; and
the first band signal is the same as the second band signal.

19. A measurement method comprising:
a body motion signal extraction step of extracting a body motion signal containing a component generated by body motion from a first band signal containing a component in a first frequency band of a first measurement signal acquired by illuminating a portion having a pulse with light of a predetermined wavelength; and
an arithmetic step of generating a pulse wave signal, the pulse wave signal being a differential signal between a second band signal and the body motion signal, the second band signal containing a component in a second frequency band of the first measurement signal.
